(19) United States
(12) Patent Application Publication
(10) Pub. No.: US 2019/0231707 A1
(43) Pub. Date: Aug. 1, 2019

(54) TRANSDERMAL DRUG DELIVERY DEVICE FOR DELIVERING OPIOIDS

(71) Applicant: CHRONO THERAPEUTICS INC., Hayward, CA (US)

(72) Inventors: Pete A. STILES, Prairie, MN (US); W. Michael HOOTEN, Rochester, MN (US); Mark Daniel SULLIVAN, Seattle, WA (US); Patrick H. RUANE, Dublin, CA (US); David J. MATLY, Oakland, CA (US); Navdeep KAUR, San Jose, CA (US); Anubhav ARORA, Hayward, CA (US); Ravi PAMNANI, Los Altos, CA (US)

(21) Appl. No.: 16/330,111

(22) PCT Filed: Sep. 28, 2017
(86) PCT No.: PCT/US17/54093

§ 371 Date: Mar. 4, 2019

Related U.S. Application Data
(60) Provisional application No. 62/401,043, filed on Sep. 28, 2016.

(51) Int. Cl. A61K 9/70; A61B 5/00; A61P 25/04; A61K 31/485; A61B 5/1171; A61B 5/1172

(52) U.S. Cl. CPC A61K 9/7023; A61B 5/4839; A61P 25/04; A61B 5/0205; A61B 5/1176; A61B 5/6833; A61B 5/1172; A61K 31/485

(57) ABSTRACT

Systems and methods are provided herein for transdermal delivery of an opioid. The opioids can be delivered in a decreasing dose (dose-tapering) manner to minimize withdrawal symptoms and abuse. The systems are also designed to limit a person besides the patient from receiving the drug with the transdermal delivery device. The systems can also include abuse-deterrence features that limit the ability of a patient or unauthorized user from tampering with the device to receive the active ingredient. Novel transdermal drug delivery formulations are also provided that include an opioid agonist or partial agonist and opioid antagonist which prevent the abuse of formulation.

### CROSS REFERENCE TO RELATED APPLICATIONS

The present application claims the benefit of U.S. Provisional Application No. 62/401,043 filed on Sep. 28, 2016, the disclosure of which is incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present application relates generally to transdermal drug delivery devices adapted to provide an opioid agonist to the user.

### BACKGROUND

Currently, there is no FDA-approved or other regulatory agency-approved wearable device for the treatment of opioid dependence or opioid addiction or chronic pain. Typical state-of-the-art in treatment of opioid dependence or chronic pain is administration of prescription medication in the form of oral pills. These are dispensed to the patient(s) as per the prescription written by the treating physician. Currently, there are limited measures in place for preventing diversion of these prescription medications once dispensed to the patient. In addition once the medication is dispensed to the patient there is no way to ensure that the patient is not taking more pills than the recommended dosage level.

Further, several forms of these prescription medications are susceptible to abuse, where patients or unauthorized users extract the active pharmaceutical ingredient (API) from the dosage form and abuse it by taking it in an alternative manner such as via injecting, snorting, etc. The alternate non-approved means of ingesting the API can result in a powerful euphoric effect of the medication experienced by the user or may prove fatal for the patient/user.

An ideal dosing profile for tapering patients off of opioids typically utilizes an asymptotic decrease in dosing that can include a sharp decrease in dose during the early phase followed by a long period of slow decrease in dose. Currently available oral pills represent a less-than-ideal dosing regimen for tapering, as they typically do not provide this asymptotic decrease since the step-down is a decrease in the number of pills taken or switching to pills with lower dosage strength. Providing opioids in pill format is also not ideal because pills are easy to divert to an unauthorized user and also easy to abuse by taking extra pills above the recommended dosage. Further, pills are not effective in keeping the patient blinded to the medication, which can be just as, if not more important than the medication itself for effective tapering. As a result, patients do not adhere to the recommended regimen for tapering off of opioids.

Improved systems and methods for delivering opioids are needed that can reduce the likelihood of abuse, reduce withdrawal symptoms, decrease divergence of the opioid, and replace the use of pills.

### SUMMARY OF THE DISCLOSURE

The present invention relates to systems and methods of transdermal drug delivery devices for providing an opioid treatment to a user. The devices may feature one or more than one form of abuse-deterrence.

Transdermal drug delivery devices are provided herein. In some embodiments the transdermal drug delivery devices can include: an active pharmaceutical ingredient comprising an opioid agonist or partial opioid agonist disposed in an opioid source; an opioid antagonist disposed in the opioid source; a transdermal drug delivery membrane configured to contact a skin of a patient and to provide the opioid agonist to the skin of the patient; a fluid communication pathway between the opioid source and the transdermal drug delivery membrane; a patient engagement surface adapted to secure the transdermal drug delivery device to the skin of the patient; and a process controller configured to control a delivery of the opioid from the opioid source to the transdermal drug delivery membrane.

In one aspect, the transdermal drug delivery devices can include: a biometric identification module configured to determine a patient biometric parameter. The biometric identification module can include one or more of: pulse oximeter, fingerprint scanner, heart rate sensor, ECG sensor, skin sensors, temperature sensor, blood flow sensor, impedance sensor, retina scanner, voice activation or recognition, and facial recognition system. In some embodiments the process controller is adapted to verify the patient biometric parameter prior to delivery of the opioid agonist or partial opioid agonist. In some embodiments, he process controller is adapted to analyze a patient biological parameter to determine a symptom associated with opioid toxicity in the patient. The process controller can be further configured to send an alert or notification wirelessly upon detection of the symptom associated with opioid toxicity in the patient.

In any of the embodiments described herein the opioid antagonist can include Naloxone, Naltrexone, Nalmefene, or Samidorphan.

In some embodiments the opioid antagonist is in a container separate from the opioid source, wherein the container is adapted to break under a tampering force to mix the opioid agonist and the opioid antagonist.

In any of the embodiments described herein the opioid antagonist can include a modified opioid antagonist. In some aspects the modified opioid antagonist includes a prodrug or a salt of the opioid antagonist. In some embodiments the modified opioid antagonist is in a solution with the opioid agonist. In some embodiments the modified opioid antagonist is in an inactive state in the transdermal drug delivery device in vitro but is configured to convert to an active state in vivo in systemic circulation in the patient.

In some embodiments the modified opioid antagonist comprises a prodrug of Naltrexone or Naloxone. In one aspect the prodrug of Naltrexone or Naloxone comprises a conjugation with poly ethylene glycol, an ester, or a carbonate. In yet another aspect the modified opioid antagonist is present in nano-particles or micro-particles of a polymer or co-polymer. The polymer or co-polymer can include one or more of: cyclodextrins, poly-ethylene glycol, poly lactic acid, poly glycolic acid, poly caprolactone, cellulose acetate phthalate, poly(lactic-co-glycolic acid), hydroxypropyl cellulose, hydroxypropyl methylcellulose, hydroxypropyl methyl cellulose phthalate, hydroxypropyl methyl cellulose acetate succinate, and gelatin.

In any of the embodiments described herein the opioid antagonist can be encapsulated in a polymer or inclusion complexes in the form of micro and/or nanoparticles.

In any of the embodiments described herein the opioid antagonist can be in an isotropic mixture of oil, water, surfactant and/or co-surfactant. In one aspect the opioid antagonist can be dispersed in a lipophilic phase.

In any of the embodiments described herein the process controller is further configured to provide an opioid antagonist to the patient upon detection of the symptom associated with opioid toxicity in the patient.

In any of the embodiments described herein the opioid source contains a plurality of doses.

In any of the embodiments described herein the processor can be further configured to provide opioid agonist to the patient according to a drug delivery regimen. In one aspect the drug delivery regimen is patient-specific and preprogrammed into the transdermal drug delivery device by a healthcare provider. In another aspect the drug delivery regimen includes a plurality of opioid doses. In yet another aspect the drug delivery regimen comprises an asymptotic daily drug dosing or drug tapering regimen. The asymptotic daily drug dosing or drug tapering regimen can include each opioid agonist dose decreasing over a previous opioid agonist dose by an amount that is lower than a decrease in opioid dosage defined by the two preceding opioid agonist doses. In another aspect the drug delivery regimen can include a high first opioid dose and a series of decreasing subsequent opioid doses. In yet another aspect the drug delivery regimen has a smooth tapered or smooth dose-titration profile.

In any of the embodiments described herein the patient engagement surface can be adapted to be worn for greater than about 1 day. In any of the embodiments described herein the patient engagement surface can be adapted to be worn for greater than about 7 days. In any of the embodiments described herein the patient engagement surface can be adapted to be worn for greater than about 14 days.

In any of the embodiments described herein the transdermal drug delivery device can include a wireless data communication module adapted to send and receive wireless data between the transdermal drug delivery device and a wireless network. In some embodiments the wireless communication module is adapted to send data corresponding to one or more of: patient compliance, tampering detection, doses delivered, days worn, and data from a biometric sensor.

In any of the embodiments described herein the opioid agonist or partial agonist is provided in a solution with a biocompatible solvent.

In any of the embodiments described herein device includes a first portion adapted to engage with the second portion to form the transdermal drug delivery device with the first portion including a device housing and the processor and the second portion including the patient engagement surface and the opioid source.

In any of the embodiments described herein the opioid agonist or partial agonist comprises one or more of: fentanyl, morphine, oxycodone, hydromorphone, tramadol, oxymorphone, alfentanil, sufentanil, methadone, buprenorphine, and hydrocodone.

In any of the embodiments described herein the transdermal drug delivery device can further include an aversive agent. In any of the embodiments described herein the transdermal drug delivery device can further include activated charcoal adapted to contact the opioid agonist when the device is contacted with a tampering force. In any of the embodiments described herein the transdermal drug delivery device can further include an oxidizing agent and/or a metabolizing agent.

In any of the embodiments described herein the opioid source includes a reservoir and the fluid communication pathway further includes a bolus reservoir adapted to receive a portion of the opioid source from the reservoir and to move the portion of the opioid source from the bolus reservoir to the transdermal drug delivery membrane.

In any of the embodiments described herein the transdermal drug delivery device includes a piston adapted to be controlled by the process controller to deliver the opioid agonist or partial agonist from the opioid source to the transdermal drug delivery membrane in a plurality of different predetermined amounts.

In any of the embodiments described herein the opioid source, transdermal drug delivery membrane, fluid communication pathway, and patient engagement surface are disposed in a disposable part and the process controller is disposed in a reusable part with the reusable part and the disposable part are adapted to be removably engaged.

In any of the embodiments described herein the devices can further include an identification module configured to receive an authentication parameter. In one aspect the identification module can be adapted to receive the authentication parameter. In another aspect the process controller cam be adapted to verify the authentication parameter prior to delivery of the opioid agonist or partial opioid agonist. In some cases the authentication parameter is a code associated with a disposable part of the transdermal drug delivery device. In yet another aspect the authentication parameter is a user specific alphanumerical code, symbol, image, or barcode.

In any of the embodiments described herein the opioid agonist can include hydrophobic droplets dispersed in a hydrophilic phase with the hydrophilic phase further including the opioid antagonist dissolved in a hydrophilic form.

Method of delivering an opioid agonist to a patient are also provided. In some embodiments the methods can include initiating a transdermal opioid delivery protocol; providing a first dose of the opioid agonist or partial agonist to the skin of the patient from a transdermal drug delivery device, wherein the transdermal drug delivery device includes an opioid antagonist; providing a second dose of the opioid agonist or partial agonist to the skin of the patient from the transdermal drug delivery device based on the transdermal opioid delivery profile; and providing a third dose of the opioid agonist or partial agonist to the skin of the patient from the transdermal drug delivery device based on the transdermal opioid delivery profile. The drug delivery protocol can include a plurality of opioid doses. The drug delivery protocol can include a treatment regimen with an asymptotic dose-tapering profile. In some aspects the asymptotic dose-tapering profile can include each opioid agonist dose decreasing over a previous opioid dose by an amount that is lower than a decrease in opioid dosage defined by the two preceding opioid agonist doses. In yet another aspect the drug delivery protocol includes a high first opioid dose and a series of decreasing opioid doses.

In some embodiments the second dose and first dose define a first percentage decrease based on a difference in the first dose and second dose divided by the first dose, the third dose and second dose define a second percentage decrease based on a difference in the second dose and third dose divided by the second dose with the first percentage decrease being equal to or greater than the second percentage decrease.

In some embodiments the patient does not know an amount of the opioid provided to the skin in any of the first, second, or third doses.

The methods can include providing any of the transdermal drug delivery device described herein.

In some embodiments the methods can further include verifying a patient identity with the transdermal drug delivery device prior to initiating the transdermal drug delivery protocol.

In one aspect verifying the patient identity is done by observing a patient biometric parameter with a biometric identification module of the transdermal drug delivery device.

In another aspect verifying the patient identity includes analyzing data obtained from one or more of a pulse oximeter, fingerprint scanner, heart rate sensor, ECG sensor, skin sensors, temperature sensor, blood flow sensor, impedance sensor, retina scanner, voice activation or recognition, and facial recognition system of the transdermal drug delivery device. In yet another aspect verifying the patient identity includes receiving an authentication parameter. The authentication parameter can be a patient-specific authentication parameter. The methods can further include generating the patient-specific authentication parameter. The methods can further include providing the patient-specific authentication parameter to the patient. The patient-specific authentication parameter can be a user-specific alphanumerical code, symbol, image, or barcode. The authentication parameter can be a code associated with a disposable part of the transdermal drug delivery device. The authentication parameter can be an alphanumerical code, symbol, image, or barcode.

In some embodiments the transdermal drug delivery protocol is pre-programmed by a healthcare provider.

In one aspect the methods include wearing the transdermal drug delivery device for greater than about 1 day. In another aspect the methods include the transdermal drug delivery device for greater than about 7 days. In yet another aspect the methods include wearing the transdermal drug delivery device for greater than about 14 days.

In some embodiments the transdermal opioid delivery protocol has a duration of greater than about two weeks. In some embodiments the transdermal opioid delivery protocol has a duration of greater than about four weeks. In some embodiments the transdermal opioid delivery protocol has a duration of greater than about eight weeks. In some embodiments the transdermal opioid delivery protocol has a duration of greater than about twelve weeks. In some embodiments the transdermal opioid delivery protocol has a duration of about two weeks to about sixteen weeks. In some embodiments the transdermal opioid delivery protocol has a duration of greater than about twenty weeks.

In any of the embodiments described herein the opioid agonist comprises one or more of: fentanyl, morphine, oxycodone, hydromorphone, tramadol, oxymorphone, alfentanil, sufentanil, methadone, buprenorphine, and hydrocodone.

In any of the embodiments described herein the transdermal opioid delivery protocol includes a dose-tapering profile that has a smooth tapered profile.

In any of the embodiments described herein the methods can further include determining a patient biological parameter to determine a symptom associated with opioid toxicity in the patient. In one aspect the method can include sending an alert or notification wirelessly upon detection of the symptom associated with opioid toxicity in the patient to a healthcare provider or emergency medical professional. The patient biological parameter can be obtained from one or more of a pulse oximeter, heart rate sensor, skin sensors, temperature sensor, blood flow sensor, impedance sensor, and retina scanner of the transdermal drug delivery device. The symptom associated with opioid toxicity in the patient can include a change in a breathing pattern, change in a heart rate, or combinations thereof. The methods can further include providing the opioid antagonist to the patient after detection of the symptom associated with opioid toxicity.

In any of the embodiments described herein the methods can include the use of a modified opioid antagonist. The modified opioid antagonist can include a prodrug, or a salt of the opioid antagonist. The opioid antagonist or modified opioid antagonist can include Naloxone, Naltrexone, Nalmefene, or Samidorphan. In one aspect the first dose of the opioid is in a solution with the modified opioid antagonist. In some cases the first dose of the opioid is absorbed transdermally through the skin of the patient and the opioid antagonist is not absorbed through the skin of the patient. In any of the embodiments described herein the transdermal drug delivery device includes a transdermal membrane that is permeable to the opioid agonist or partial agonist with the opioid antagonist being substantially impermeable to the transdermal membrane. In one aspect the modified opioid antagonist can include a prodrug of Naltrexone or Naloxone. The prodrug of Naltrexone or Naloxone can include a conjugation with poly ethylene glycol, an ester, or a carbonate. In yet another aspect the modified opioid antagonist is present in nano-particles or micro-particles comprising a polymer. The polymer can include one or more of: cyclodextrins, poly-ethylene glycol, poly lactic acid, poly glycolic acid, poly caprolactone, cellulose acetate phthalate, poly(lactic-co-glycolic acid), hydroxypropyl cellulose, hydroxypropyl methylcellulose, hydroxypropyl methyl cellulose phthalate, hydroxypropyl methyl cellulose acetate succinate, and gelatin. In yet another aspect the opioid antagonist can be encapsulated in a polymer or inclusion complexes in the form of micro and/or nanoparticles. In some cases the opioid antagonist is in an isotropic mixture of oil, water, surfactant and/or co-surfactant. In another aspect the opioid antagonist is dispersed in a lipophilic phase.

In some embodiments the methods the first dose is provided by a first transdermal drug delivery device, the second dose is provided by a second transdermal drug delivery device, and the third dose is provided by a third transdermal drug delivery device.

In some embodiments the methods the transdermal drug delivery device further includes an opioid source containing the opioid agonist or partial opioid agonist and the opioid antagonist, a transdermal drug delivery membrane configured to contact a skin of a patient and to provide the opioid agonist to the skin of the patient; a fluid communication pathway between the opioid source and the transdermal drug delivery membrane; a patient engagement surface adapted to secure the transdermal drug delivery device to the skin of the patient; and a process controller configured to control a delivery of the opioid from the opioid source to the transdermal drug delivery membrane. The opioid source can include a reservoir and the fluid communication pathway further comprising a bolus reservoir adapted to receive a portion of the opioid source from the reservoir and to move the portion of the opioid source from the bolus reservoir to the transdermal drug delivery membrane. The transdermal drug delivery device can include a piston adapted to be controlled by the process controller to deliver the opioid agonist or partial agonist from the opioid source to the transdermal drug delivery membrane in a plurality of different predetermined amounts.

In one aspect the methods described herein can include any of the transdermal drug delivery devices described herein.

Methods for delivering an opioid to a patient are provided. The methods can include moving an active pharmaceutical ingredient comprising an opioid agonist or partial agonist disposed in an opioid source from the opioid source to a transdermal membrane of a transdermal drug delivery device; moving a modified opioid antagonist disposed in the opioid source from the opioid source to the transdermal membrane; and passing a first portion of the active pharmaceutical ingredient comprising the opioid agonist or partial agonist across the transdermal membrane.

Passing the first portion of the active pharmaceutical ingredient comprising the opioid agonist or partial agonist across the transdermal membrane can be at a first flux rate. In one aspect the modified opioid antagonist has a second flux rate across the transdermal membrane. In some cases the second flux rate is about zero. In some embodiments a ratio of the second flux rate to the first flux rate is less than about 1:10. In some embodiments a ratio of the second flux rate to the first flux rate is less than about 1:25. In some embodiments a ratio of the second flux rate to the first flux rate is less than about 1:50. In some embodiments a ratio of the second flux rate to the first flux rate is less than about 1:100.

The methods can further include passing a second portion of the opioid agonist or partial agonist across a skin of a patient wearing the transdermal drug delivery device at a first absorption rate. In one aspect the methods further include passing a portion of the modified opioid antagonist across the skin of the patient wearing the transdermal drug delivery device at a second absorption rate. In some cases the second absorption rate is about zero. In some embodiments a ratio of the second absorption rate to the first absorption rate is less than about 1:10. In some embodiments a ratio of the second absorption rate to the first absorption rate is less than about 1:25. In some embodiments a ratio of the second absorption rate to the first absorption rate is less than about 1:50. In some embodiments a ratio of the second absorption rate to the first absorption rate is less than about 1:100.

In any of the methods described herein the modified opioid antagonist can include a prodrug or a salt of an opioid antagonist. In one aspect the modified opioid antagonist is in a solution with the opioid agonist or partial agonist. In another aspect the modified opioid antagonist is in an inactive state in the transdermal drug delivery device in vitro but is configured to convert to an active state in vivo in systemic circulation in the patient. In yet another aspect the modified opioid antagonist comprises a prodrug of Naltrexone or Naloxone. The prodrug of Naltrexone or Naloxone can include a conjugation with poly ethylene glycol, an ester, or a carbonate. In some embodiments the modified opioid antagonist is present in nano-particles or micro-particles of a polymer or co-polymer. The polymer or co-polymer can include one or more of: cyclodextrins, poly-ethylene glycol, poly lactic acid, poly glycolic acid, poly caprolactone, cellulose acetate phthalate, poly(lactic-co-glycolic acid), hydroxypropyl cellulose, hydroxypropyl methylcellulose, hydroxypropyl methyl cellulose phthalate, hydroxypropyl methyl cellulose acetate succinate, and gelatin.

In some embodiments methods are provided. The methods can include receiving an opioid treatment regimen for a patient; generating a patient-specific code for the patient; receiving the patient-specific code; and initiating a transdermal opioid delivery protocol with a transdermal drug delivery device based on the opioid treatment regimen. The methods can further include receiving the patient-specific code from the transdermal drug delivery device. The methods can further include receiving the patient-specific code from a wireless data transfer from a remote computer network or a smartphone application. The methods can further include receiving a code associated with a disposable drug cartridge of the transdermal drug delivery device. The code associated with the disposable drug cartridge can be an alphanumerical code, symbol, image, or barcode. The methods can further include receiving a code associated with a packing of a disposable drug cartridge of the transdermal drug delivery device. The code associated with the packing of the disposable drug cartridge can be an alphanumerical code, symbol, image, or barcode. The methods can further include verifying the patient identity by observing a patient biometric parameter with a biometric identification module of the transdermal drug delivery device. The methods can further include verifying the patient identity by analyzing data obtained from one or more of a pulse oximeter, fingerprint scanner, heart rate sensor, ECG sensor, skin sensors, temperature sensor, blood flow sensor, impedance sensor, retina scanner, voice activation or recognition, and facial recognition system of the transdermal drug delivery device. The transdermal drug delivery protocol can pre-programmed by a healthcare provider. The methods can include using any of the transdermal drug delivery devices described herein. The transdermal drug delivery protocol can include any of the transdermal drug delivery protocols described herein.

The methods can further include analyzing one or more parameters associated with a patient compliance to the transdermal opioid delivery protocol and/or the opioid treatment regimen. The methods can further include generating a compliance data based on the one or more parameters associated with the patient compliance to the transdermal opioid delivery protocol and/or the opioid treatment regimen. The methods can further include providing the compliance data to the healthcare provider. Providing the compliance data can include sending an electronic message, electronic notification, or providing a graphical user interface containing the compliance data. Providing the compliance data can include incorporating the compliance data into an electronic medical record for the patient.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A illustrates an embodiment of a transdermal drug delivery device for delivering an opioid.

FIG. 1B is a schematic illustration of an embodiment of a transdermal drug delivery device for delivering an opioid.

FIGS. 2A and 2B illustrate embodiments of transdermal drug delivery devices for delivering an opioid that contain an antagonist.

FIG. 3 illustrates a transdermal drug delivery device with a sensor for analyzing biometric data associated with the wearer of the device in accordance with some embodiments.

FIG. 4A illustrates a transdermal drug delivery device in accordance with some embodiments.

FIG. 4B illustrates a schematic example of a portion of a transdermal drug delivery device in accordance with some embodiments.

FIG. 5A illustrates a transdermal drug delivery device in accordance with some embodiments.

FIG. 5B illustrates a schematic example of a portion of a transdermal drug delivery device in accordance with some embodiments.

FIGS. 6A and 6B illustrate an example of an asymptotic dose-tapering profile that can be used with the methods and systems described herein.

FIG. 7 illustrates transdermal drug delivery devices for delivering different doses of an opioid agonist in accordance with some embodiments.

FIG. 8 illustrates an embodiment of a transdermal drug delivery device for delivering an opioid with a biometric sensor.

FIG. 9 illustrates an embodiment of a transdermal drug delivery device for delivering an opioid with a biometric sensor along with schematic examples of biometric confirmation.

FIG. 10 illustrates an embodiment of a transdermal drug delivery device for delivering an opioid with an integrated sensing technology.

FIG. 11 illustrates an embodiment of a transdermal drug delivery device for delivering an opioid.

FIG. 12 illustrates a display showing information related to patient compliance in accordance with some embodiments.

FIGS. 13A-13C illustrate chemical formulas of Naltrexone (abbreviated as “NTX” in some places) and Naloxone (abbreviated as “NLX” in some places) with various conjugations that can be used in the transdermal drug delivery systems described herein.

FIG. 14 illustrates chemical formulas of Naltrexone and Naloxone conjugated with poly ethylene glycol (PEG) that can be used in the transdermal drug delivery systems described herein.

FIG. 15 is a graph illustrating the average cumulative delivery across a cadaver skin for Naltrexone and PEG conjugated Naltrexone.

FIG. 16 is a graph comparing the release profile for pure Naltrexone versus Naltrexone delivered with nanoparticles in accordance with some embodiments.

### DETAILED DESCRIPTION

Opioid abuse and overdosing are huge public health issues. Many patients that are prescribed opioids to treat acute pain can develop addictions and/or suffer from withdrawal symptoms when stopping treatment. Furthermore, providing opioids as pills makes it easy for the patient to take additional pills above the prescribed amount. Pills are also easy to divert to an unauthorized user. One goal of the present disclosure is to provide an opioid delivery system that eliminates the need for pills. For example, an opioid can be provided within a transdermal drug delivery device. The opioid can be absorbed passively through the skin of the patient.

Providing opioids in the form of pills, patches, or other formats that can be self-administered have little or no safeguards in place to prevent the user from taking extra medication above the recommended or safe dosing level. Providing pills, patches, or other formats with opioids also can allow for off-label ingestion of the opioid through snorting, injecting, or contacting with mucous membranes. Ingestion of the opioid through snorting, injecting, or contacting with mucous membranes can provide an euphoric effect to the user that can increase the chances of developing an addiction and may prove fatal as well. Another goal of the present disclosure is to provide the opioid medication in a format that limits or eliminates the possibility of ingesting the opioid in an off-label manner, such as through intravenous injection, snorting, contacting with mucous membranes, etc.

The transdermal drug delivery devices described herein can include diversion control and abuse prevention features. For example, a biometric sensor can be used to limit divergence of the opioid medication. In some embodiments the transdermal delivery devices can include a biometric sensor that can be used to verify that the person wearing the device is the patient with the prescribed opioid treatment.

The devices described herein can also utilize additional abuse deterrence and anti-tampering features. For example, the transdermal opioid drug delivery devices described herein can be provided with an opioid antagonist to limit abuse or off-label ingestion. The opioid antagonist can be arranged within the transdermal drug delivery device such that it is released or contacts the opioid when the device is tampered with by a user. The inclusion of the opioid antagonist with the opioid agonist limits the effect of opioid agonist in the abuser and thereby deters abuse. For example, the opioid formulation can contain an opioid agonist such as fentanyl, or a partial agonist such as buprenorphine in a free-base form which will allow its passage through the skin. Specifically, the opioid agonist can be hydrophobic/lipophilic with Log P value between 1 and 5 (Log P refers to the lipophilicity of non-ionized species. Lipophilicity represents the affinity of a molecule or a moiety for a lipophilic environment. It is commonly measured by its distribution in a biphasic system (e.g. partition coefficient in octanol-water-Pure & Appl. Chem., Vol. 70, No. 5, pp. 1129-1143, 1998). Typical candidate drugs for transdermal delivery such as opioid agonists have Log P value between 1 and 5. The abuse deterrent formulation may contain a solid or liquid preparation of opioid antagonists such as naloxone or naltrexone in a form that closely mimics the physicochemical properties of the opioid agonist (e.g. similar log P or hydrophobicity). Tampering with the drug cartridge will result in mixing of opioid agonist and antagonist. Since both active pharmaceutical ingredients (APIs) will have similar physicochemical properties, it would prevent separation or extraction of the opioid agonist if the cartridge is busted open, subjected to simple household solvents or tampered with simple procedures.

In some cases the opioid antagonist can be mixed in a solution with the opioid agonist such that the opioid antagonist is not absorbed through the skin during normal use of the device but the mixture of the opioid antagonist and opioid agonist would prevent an unauthorized user from abusing the mixture of opioid antagonist and opioid agonist. For such embodiments, the opioid agonist (e.g. Fentanyl) and opioid antagonist or a modified antagonist (e.g. naltrexone) will be present as a mixture in the formulation. The opioid agonist may be present in a form that can be delivered via transdermal route upon formulation exposure to the skin (e.g. free-base form). The opioid antagonist may be present in a form that is not readily permeable across skin compared to the opioid agonist, e.g. a modified form such as a salt form or an analog such as ester of the opioid antagonist which allows for different physicochemical properties than the opioid agonist in the formulation solvent system. The modified form would be such that extraction of this co-mixed formulation via typical methods of tampering, e.g. extraction with common household solvents, would result in substantial partitioning of both the opioid agonist and the opioid antagonist in the extraction solvent, thus preventing opioid abuse.

Opioids are generally used for treating chronic pain. However, a phenomenon called opioid-induced hyperalgesia (OIH) may make opioid pills a poor way to treat patients with chronic pain (A Comprehensive Review of Opioid-Induced Hyperalgesia by Marion Lee et al. 2011). Opioid-induced hyperalgesia is defined as a state of nociceptive sensitization caused by exposure to opioids. The condition is characterized by a paradoxical response whereby a patient receiving opioids for the treatment of pain could actually become more sensitive to certain painful stimuli. Opioid-induced hyperalgesia can make opioids better for treating acute pain than treating chronic pain. In addition, counterintuitively, studies have found that increasing the opioid dosage for a patient can actually cause the patient to perceive increased levels of pain from the same/similar stimuli. The psychological and physiological responses, such as withdrawal, opioid-induced hyperalgesia, etc., in patients receiving an opioid for treating pain can lead to the development of an addiction. The physiological and psychological responses can lead to a self-reinforcing cycle where the patient increases the opioid dosage to treat the perceived increase in pain and instead perceives additional pain, which leads to further opioid dosage increases. The self-reinforcing cycle can further increase patient tolerance of the opioid and lead to potentially fatal overdosing. Conversely, decreasing the opioid dosage can lead to intense withdrawal effects in the patient that make it hard to decrease or discontinue taking the opioid.

The self-reinforcing cycle to increase dosage and the withdrawal symptoms associated with decreasing dosages make it difficult for a patient to step down the dosages by taking fewer pills or pills with less active ingredient. In addition, when the user knows the dosage of the pills and has access to additional pills it can be difficult to step down the opioid dosage to discontinue the treatment. When the user knows that the dosage is stepped down on the opioid, they can perceive increased pain or the need for additional opioid. All of these factors make it difficult to effectively step down the opioid dosage with the discrete doses of opioids that are provided by pills and patches.

The transdermal drug delivery devices described herein can be used to deliver a precisely tailored dosage pattern of the opioid agonist to the skin of the patient. The drug delivery protocol or regimen can taper the opioid dosage to the patient. Another advantage is that the patient may not know the specific dosage of the opioid that is delivered with each dose or each day, thus keeping the patient blinded to the tapering protocol. The use of pills and patches let the user know the specific dosage that is delivered with each pill or patch. The patient can violate the desired tapered dosage by administering extra pills or patches. The patient not knowing the dosage of the opioid makes it much easier and more effective to taper the opioid dosage in the drug delivery regimen. For example, the use of blind dosing may reduce the likelihood of the occurrence of opioid-induced hyperalgesia and reduce any other psychological factors associated with the use of opioids. The smooth tapered dosage profile can reduce the likelihood of withdrawal symptoms and other physiological responses from occurring in the patient. The use of blind dosing and a smooth tapered profile can minimize both physiological and psychological factors associated with opioid use and opioid addiction. There is plethora of evidence in the literature supporting placebo effect in the treatment of pain. (Zubieta, J. et al. Journal of Neuroscience 24 Aug. 2005, 25 (34) 7754-7762)

The transdermal drug delivery devices described herein can be used for various applications. In some embodiments the devices can be used to treat a person addicted to opioids by providing a tapered dosing pattern that can reduce or minimize withdrawal symptoms occurring in the patient. Another example application includes providing the device to a patient after an injury, surgical procedure, or other painful event to provide a discreet treatment for acute or chronic pain associated with such an event. The patient can use the device to treat the acute or chronic pain without knowing the opioid dosage for a multi-week treatment for the acute or chronic pain. In yet another example, the devices described herein can be used by patients undergoing hospice care to provide precise opioid doses while minimizing the likelihood of an overdose and reducing the likelihood of diversion.

The transdermal drug delivery systems can include an active pharmaceutical ingredient (API) comprising an opioid agonist disposed in an opioid source, a transdermal drug delivery membrane configured to contact a skin of a patient and to provide the opioid agonist to the skin of the patient, a fluid communication pathway between the opioid source and the transdermal drug delivery membrane, a patient engagement surface adapted to secure the transdermal drug delivery device to the skin of the patient, and a process controller configured to control the delivery of opioid agonist from the opioid source to the transdermal drug delivery membrane.

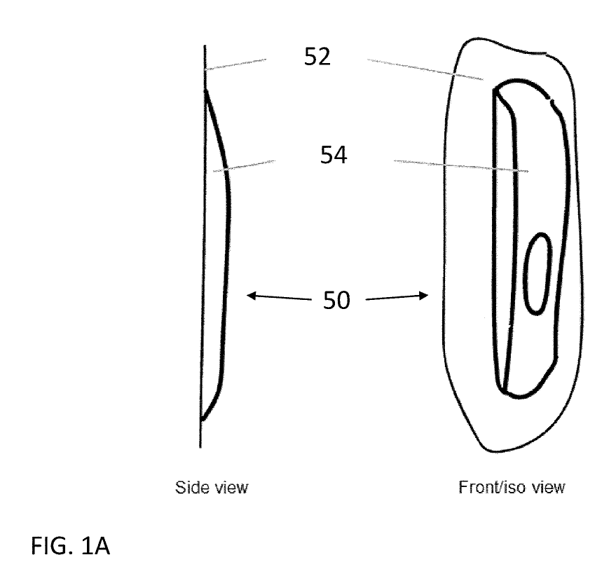

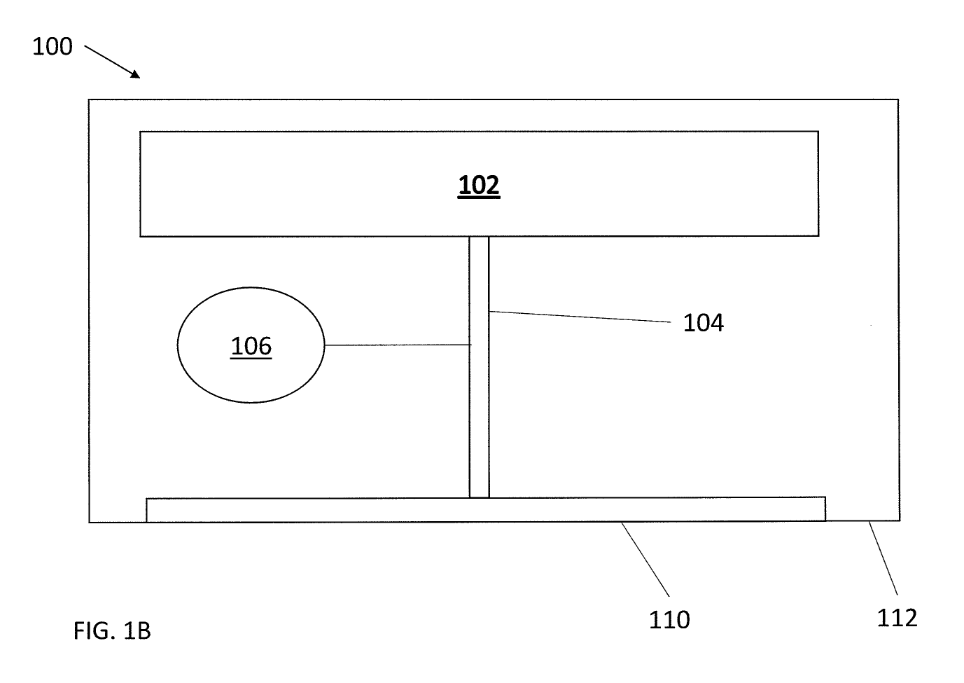

FIG. 1A illustrates an embodiment of a transdermal drug delivery device 50 for delivering an opioid agonist. The transdermal drug delivery device 50 includes an adhesive portion 52 to adhere to the skin of the patient. The transdermal drug delivery device 50 includes a housing 54 that encloses and supports the opioid source, a fluid communication pathway between the opioid source and a transdermal drug delivery membrane, and the transdermal drug delivery membrane.

FIG. 1B is a schematic illustration of an embodiment of a transdermal drug delivery device 100 for delivering an opioid. The transdermal drug delivery device 100 includes an opioid source 102 illustrated as a reservoir adapted to hold the formulation containing opioid agonist. The opioid source 102 can include one or more reservoirs or containers that hold a plurality of opioid agonist doses. The opioid source 102 can provide each of the plurality of opioid agonist doses from a single reservoir or each of the opioid agonist doses can be provided from smaller discrete containers within the opioid source 102. The smaller discrete containers can be adapted to each hold a single opioid agonist dose. The transdermal drug delivery device 100 includes a fluid communication pathway 104 that provides a conduit between the opioid source 102 and the transdermal drug delivery membrane 110. The flow of the opioid agonist from the opioid source 102 through the fluid communication pathway 104 to the transdermal membrane 110 can be controlled by a processor 106. The processor 106 can send a signal to a controller, valve, micro-pump, or other structure that facilitates the flow of opioid from the opioid source 102 through the fluid communication pathway 104 to the transdermal membrane 110. The opioid agonist diffuses across the transdermal membrane 110 into contact with the skin of the patient and can then be absorbed through the skin. The transdermal drug delivery device 100 also includes an adhesive 112 adapted to hold the transdermal drug delivery device 100 to the skin of the patient for the duration of the treatment.

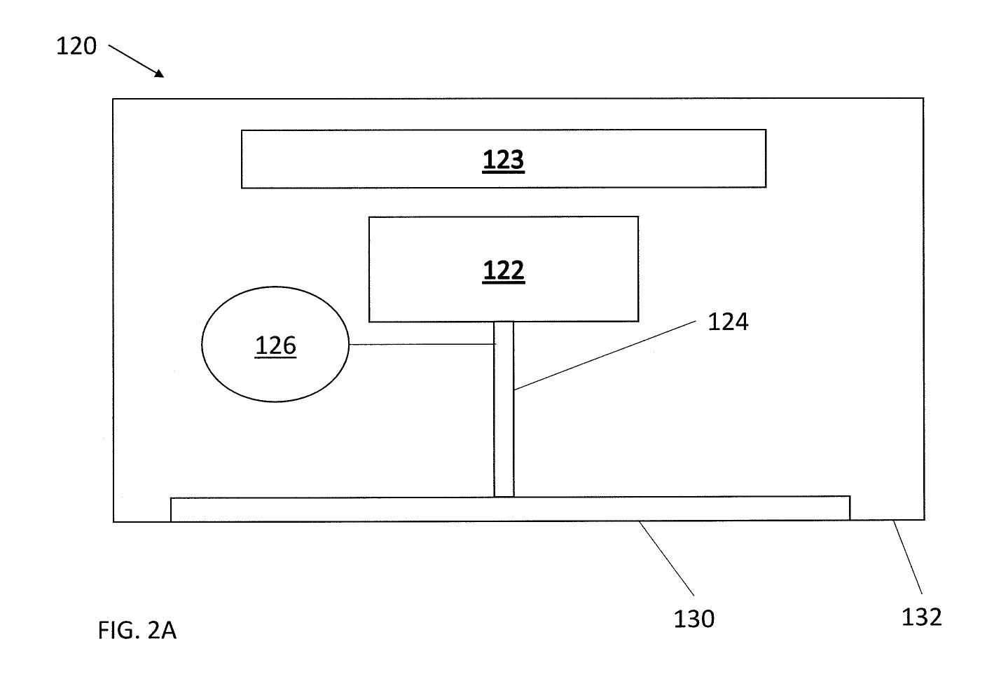

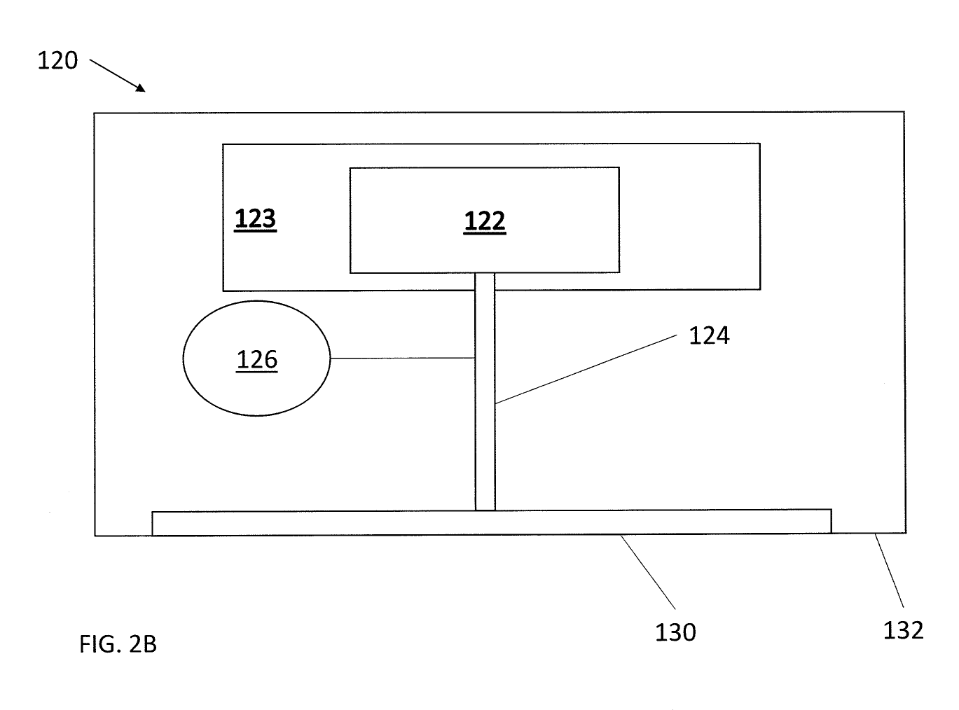

FIGS. 2A and 2B are schematic illustrations of embodiments of a transdermal drug delivery device 120 for delivering an opioid agonist. The transdermal drug delivery device 120 includes an opioid source 122 illustrated as a reservoir adapted to hold the opioid agonist. The opioid source 122 can include one or more reservoirs or containers that hold a plurality of opioid agonist doses. The device 120 includes an opioid antagonist 123 in a reservoir. FIG. 2A illustrates the opioid antagonist 123 in a reservoir adjacent to the opioid source 122. While FIG. 2B illustrates the opioid antagonist in a reservoir surrounding the opioid source 122. The opioid antagonist 123 is adapted to release the opioid antagonist if the device is tampered with such that the opioid antagonist 123 mixes with the opioid source 122 to reduce the ability to abuse the opioid source 122. The opioid source 122 can provide each of the plurality of opioid agonist doses from a single reservoir or each of the opioid agonist doses can be provided from smaller discrete containers within the opioid source 122. The smaller discrete containers can be adapted to each hold a single opioid agonist dose. The transdermal drug delivery device 120 includes a fluid communication pathway 124 that provides a conduit between the opioid source 122 and the transdermal drug delivery membrane 130. The flow of the opioid agonist from the opioid source 122 through the fluid communication pathway 124 to the transdermal membrane 130 can be controlled by a processor 126. The processor 126 can send a signal to a controller, valve, micro-pump, or other structure that facilitates the flow of opioid agonist from the opioid source 122 through the fluid communication pathway 124 to the transdermal membrane 130. The opioid agonist diffuses across the transdermal membrane 130 into contact with the skin of the patient and can then be absorbed through the skin. The transdermal drug delivery device 120 also includes an adhesive 132 adapted to hold the transdermal drug delivery device 120 to the skin of the patient for the duration of the treatment.

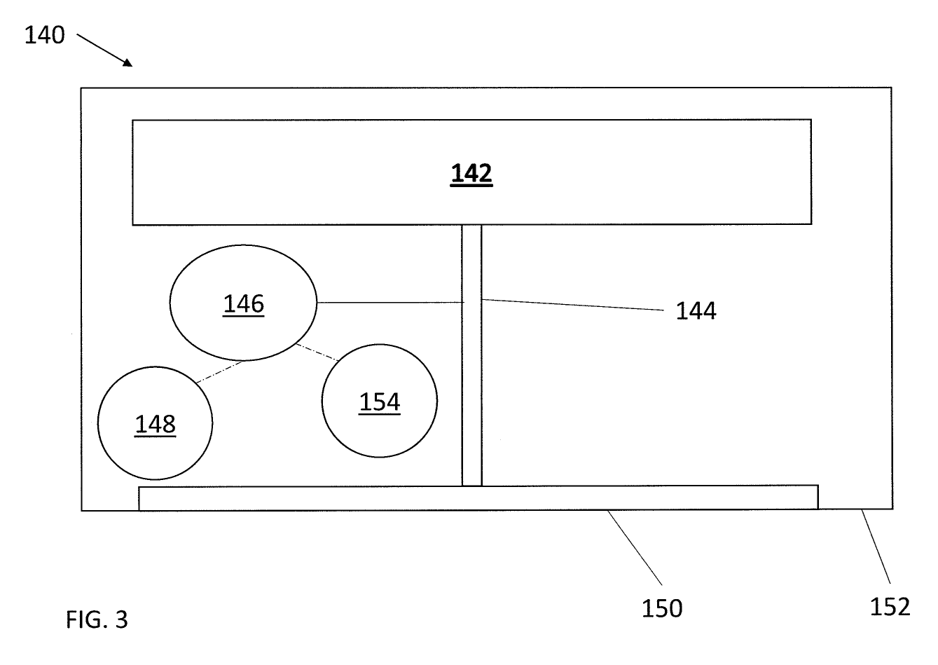

FIG. 3 is a schematic illustration of an embodiment of a transdermal drug delivery device 140 for delivering an opioid agonist. The transdermal drug delivery device 140 includes an opioid source 142 illustrated as a reservoir adapted to hold the opioid agonist. The opioid source 142 can include one or more reservoirs or containers that hold a plurality of opioid agonist doses. The opioid source 142 can provide each of the plurality of opioid agonist doses from a single reservoir or each of the opioid agonist doses can be provided from smaller discrete containers within the opioid source 142. The smaller discrete containers can be adapted to each hold a single opioid agonist dose. The transdermal drug delivery device 140 includes a fluid communication pathway 144 that provides a conduit between the opioid source 142 and the transdermal drug delivery membrane 150. The flow of the opioid from the opioid source 142 through the fluid communication pathway 144 to the transdermal membrane 150 can be controlled by a processor 146. The processor 146 can send a signal to a controller, valve, micro-pump, or other structure that facilitates the flow of opioid agonist from the opioid source 142 through the fluid communication pathway 144 to the transdermal membrane 150. The opioid agonist diffuses across the transdermal membrane 150 into contact with the skin of the patient and can then be absorbed through the skin. The transdermal drug delivery device 140 also includes an adhesive 152 adapted to hold the transdermal drug delivery device 140 to the skin of the patient for the duration of the treatment. The device 140 includes a wireless communication module 154 adapted to wirelessly transmit and receive data between the processor 146 and a computer network or device that is external to the device 140. The illustrated device 140 includes a sensor 148 that can be used to verify biometric information relating to the patient to confirm that the person wearing the device 140 is the patient for which the opioid treatment has been prescribed. The sensor 148 can also be configured to monitor other biological characteristics of the user, such as the heart rate, temperature, breathing pattern, etc. In some embodiments the opioid agonist and solvent are provided to a space between the transdermal membrane 150 and a vapor permeable membrane. The solvent can evaporate and pass through the vapor permeable membrane to increase the concentration of the opioid agonist and increase the flux of the opioid agonist across the transdermal membrane 150 and into contact with the skin.

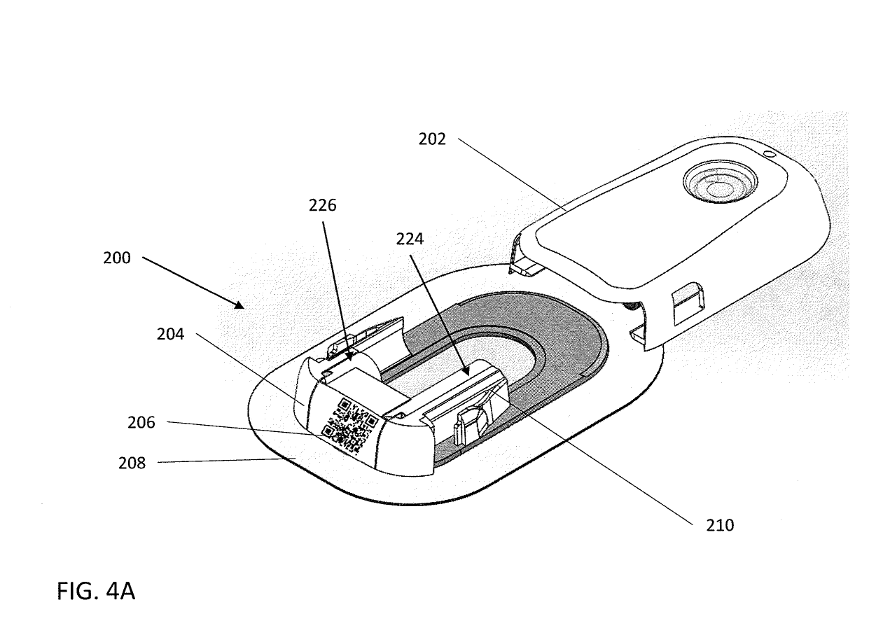

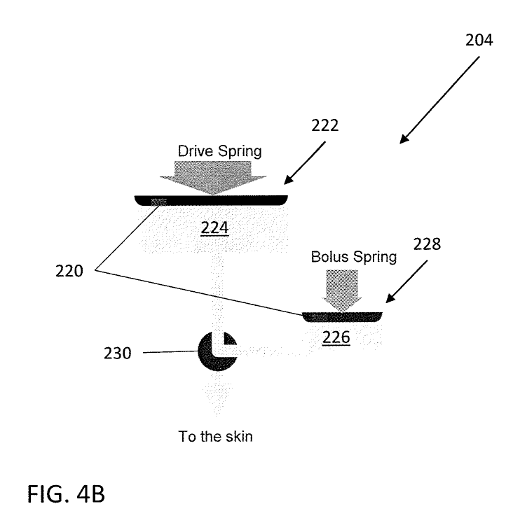

FIG. 4A illustrates a transdermal drug delivery device 200 in accordance with some embodiments. The device 200 includes a reusable part 202 and a disposable part 204. The reusable part can include the electronics and controller while the disposable part 204 can include the drug reservoir 224, optional bolus reservoir 226, transdermal membrane, adhesive, etc. The device 200 can include a QR code 206 or other scanned code on the disposable part 204 for the user to scan with a hand held computer device as part of the authentication process. After the authentication is done the controller for the reusable part 202 can be activated to proceed with the drug delivery protocol. The device 200 also includes an impedance sensor 208 to detect contact with the skin of the user. The disposable part 204 can include a monitoring sensor 210 on the drug reservoir to detect tampering with the chamber that can occur with physical tampering. Upon detecting physical tampering, the sensors 210 can send a message to the physician or other healthcare provider as part of the compliance measurements. The use of a hard plastic reservoir to contain the opioid agonist can make physical tampering more difficult. Other types of sensors can be used as well. For example the sensors can detect access such as a puncture through the sensor when a membrane sensor is used or a sudden change in volume or pressure with the reservoir. Other types of sensors can be used such as an RFID sensor, magnetic sensor for proximity or volume displacement, or other sensors.

FIG. 4B illustrates a schematic example of a portion of a disposable part 204 of a transdermal drug delivery device 200 in accordance with some embodiments. The disposable part 204 include a magnetic sensor 220 on an opioid reservoir piston 222 in the opioid reservoir 224 and a bolus reservoir piston 228 in the bolus reservoir 226. The magnetic sensors 220 can communicate with the reusable part 202 to provide information about the piston positioning and opioid delivery. The opioid source can travel from the opioid reservoir 224 through a valve 230 into the bolus reservoir 228. The opioid source can then travel from the bolus reservoir through the valve 230 and through a transdermal membrane and then into contact with the skin.

In some embodiments the transdermal drug delivery device can be configured to deliver different amounts of the opioid agonist or partial opioid agonist. For example, the transdermal drug delivery device can include a piston adapted to be controlled by the process controller to deliver the opioid agonist or partial agonist from the opioid source to the transdermal drug delivery membrane in a plurality of different predetermined amounts. The piston can be moved in a controlled fashion corresponding to the amount of opioid agonist or partial agonist expelled from the reservoir and provided to the transdermal membrane. The volume of the opioid agonist or partial agonist can vary based on the desired dose to be provided to the patient. Examples of typical dosage volumes can be about 200 μL, 150 μL, 125 μL, 100 μL, 75 μL, 50 μL, 25 μL, 10 μL, etc.

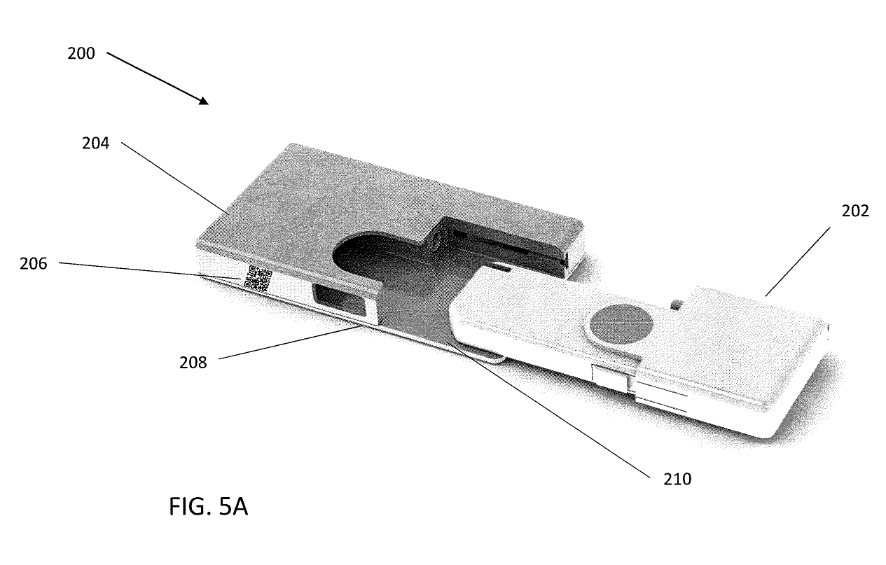

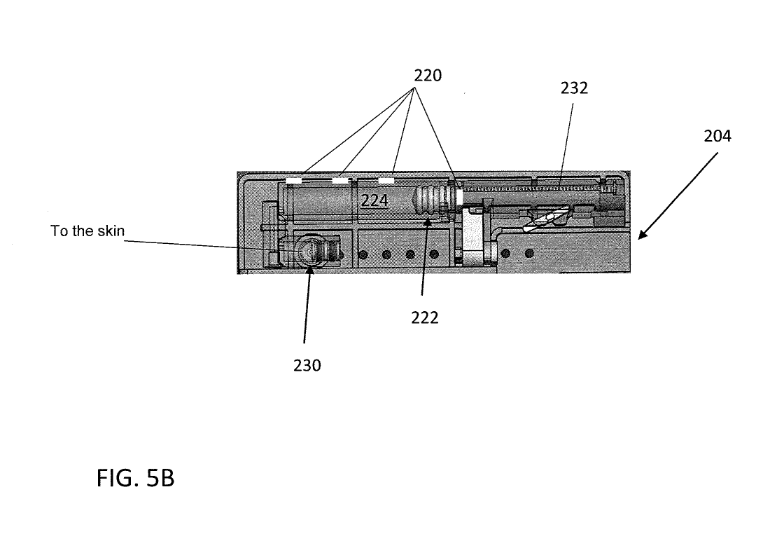

FIGS. 5A-5B illustrate a transdermal drug delivery device 200 with a single opioid reservoir in accordance with some embodiments. The device 200 includes a reusable part 202 and a disposable part 204. The reusable part can include the electronics and controller while the disposable part 204 can include a drug reservoir 224, transdermal membrane, adhesive, etc. The device 200 can include a QR code 206 or other scanned code on the disposable part 204 for the user to scan with a hand held computer device as part of the authentication process. After the authentication is done the controller for the reusable part 202 can be activated to proceed with the drug delivery protocol. The device 200 also includes an impedance sensor 208 to detect contact with the skin of the user. The disposable part 204 can include a monitoring sensor 210 on the drug reservoir to detect tampering with the chamber that can occur with physical tampering.

FIG. 5B illustrates a cross-sectional view of a portion of a disposable part 204 of a transdermal drug delivery device 200 in accordance with some embodiments. The disposable part 204 include a magnetic sensor 220 on an opioid reservoir piston 222 and in the opioid reservoir 224. The magnetic sensors 220 can communicate with the reusable part 202 to provide information about the piston positioning and opioid delivery. A piston spring 232 can provide a force to the opioid reservoir piston 222 to expel a controlled amount of the opioid source. The opioid source can travel from the opioid reservoir 224 through a valve 230 through a transdermal membrane and then into contact with the skin. The linear travel of the piston 222 can be correlated to the volume of the opioid source expelled from the opioid reservoir 224. The linear motion of the opioid reservoir piston 222 can be controlled to deliver a variable amount of the opioid source.

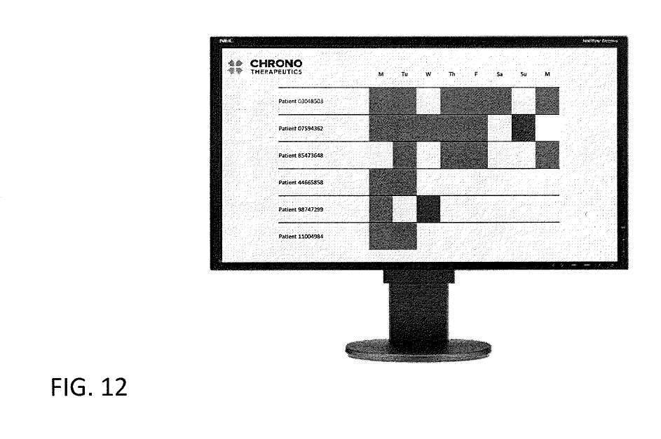

FIG. 12 illustrates a display showing information related to patient compliance in accordance with some embodiments. The display can show whether the patient has followed the recommended treatment protocol or if there has been any tampering or violations of the treatment protocol. The physician or healthcare can track the compliance through the software application. The compliance can also seamlessly integrate with the patient medical records. A companion smartphone application can also provide behavior support and reminders to the patient receiving the opioid agonist.

The transdermal drug delivery device provides the opioid agonist to the skin of the patient. The opioid agonist can then be absorbed through the skin of the patient to enter the blood stream of the patient. Transdermal drug delivery is slower than drug delivery through other means, such as injection, snorting, contact with mucous membranes, etc. Transdermal delivery of opioid can reduce the euphoric effect of opioid agonist compared to other drug delivery methods, which can also limit the likelihood of addiction from the opioid treatment.

The opioid source includes a plurality of doses of opioid agonist. For example, the plurality of doses can include enough opioid agonist doses for the prescribed opioid drug delivery regimen. The plurality of opioid agonist doses can be included in a solution, with the transdermal drug delivery device delivering different discrete volumes based on the opioid drug delivery regimen. For example, a micro-pump or other fluid delivery structure can be used to deliver the precise amount of the opioid agonist to the skin of the patient based on the patient-specific drug delivery protocol. The plurality of doses can also be arranged separately and discretely within the transdermal drug delivery device. In this example, the separate and discrete doses could be accessed according to the drug delivery regimen to deliver the material in the discrete dose to the skin of the patient.

The processor of the transdermal drug delivery device is programmed to deliver the drug delivery regimen to the patient. The drug delivery regimen is typically patient-specific and preprogrammed by a medical professional. The programming can be done through wired or wireless communication to the transdermal drug delivery device, or a cartridge or specific transdermal drug delivery device having the desired arrangement of the plurality of doses can be selected and provided to the patient.

The opioid agonist in the transdermal drug delivery device can be provided in a solution with a biocompatible solvent. The processor of the transdermal drug delivery device can control the flow of the opioid agonist from the opioid source through a fluid communication pathway to the transdermal drug delivery membrane. The transdermal drug delivery membrane can be selected with properties to facilitate the diffusion of the opioid agonist across the membrane and into contact with the skin of the patient. In some embodiments, the transdermal drug delivery membrane can limit the diffusion of any biocompatible solvent across the membrane. In some embodiments the transdermal drug delivery membrane can limit the diffusion of any opioid antagonist (optionally included within the opioid source) across the membrane. The transdermal membrane material can be selected to be compatible with the skin and to have the desired chemical properties to allow for diffusion of the opioid agonist across the membrane. Examples of the chemical properties include the membrane material, porosity, hydrophobicity/hydrophilicity, chemical treatments, etc. In some embodiments the transdermal drug delivery device can improve the flux of the opioid agonist across the membrane by removing liquid phase solvent from the membrane surface to maintain a higher concentration of the opioid at the membrane.

The transdermal drug delivery device can be designed to be worn by the patient for the duration of the opioid drug treatment regimen. The patient engagement surface of the transdermal drug delivery device can include an adhesive configured for short-term or long term wear. A band or other mechanical structure can also be used to secure the device to the user instead of an adhesive or in combination with the adhesive. In some embodiments the patient engagement surface is adapted to be worn for greater than about 1 day. In some embodiments the patient engagement surface is adapted to be worn for greater than about 7 days. In some embodiments the patient engagement surface is adapted to be worn for greater than about 14 days.

The length of the transdermal opioid delivery protocol can be tailored for the specific patient. In some embodiments, multiple transdermal drug delivery devices can be used over the duration of the treatment. In some embodiments, multiple transdermal drug delivery devices can be worn to provide a treatment for greater than about 30 days. In some embodiments multiple transdermal drug delivery devices can be worn to provide a treatment for greater than about 60 days. In some embodiments multiple transdermal drug delivery devices can be worn to provide a treatment for greater than about 90 days. In some embodiments the transdermal opioid delivery protocol can have a duration of greater than about two weeks. In some embodiments the transdermal opioid delivery protocol can have a duration of greater than about four weeks. In some embodiments the transdermal opioid delivery protocol can have a duration of greater than about eight weeks. In some embodiments the transdermal opioid delivery protocol can have a duration of greater than about twelve weeks. In some embodiments the transdermal opioid delivery protocol can have a duration of about two weeks to about sixteen weeks. In some embodiments the transdermal opioid delivery protocol can have a duration of about two weeks to about twenty weeks. In some embodiments the transdermal opioid delivery protocol can be longer than about 20 weeks.

The transdermal drug delivery device can include a wireless data communication module adapted to send and receive wireless data between the transdermal drug delivery device and a wireless data network. For example, the wireless communication module can send data corresponding to one or more of the following: patient compliance, doses delivered, days worn, tampering, and data from any of the sensors on board the device.

The transdermal drug delivery device can include two or more discrete pieces that can removably engage with each other. For example, a disposable drug cartridge or disposable part can be used with a reusable electronics portion. The drug delivery regimen could be provided to the patient using multiple disposable cartridges. The reusable portion can include a device housing and the processor and the disposable portion can include the patient engagement surface, the opioid agonist within the opioid source, a transdermal drug delivery membrane, and a fluid communication pathway between the opioid source and the transdermal drug delivery.

The transdermal drug delivery devices described herein can be used to provide an entire pain treatment regimen to the patient. In one example, the patient is identified as needing a pain management treatment, such as after an injury or surgical procedure. The healthcare provider can determine an appropriate opioid dosing level, dosing schedule, and tapering schedule. The dosing schedule can either be programmed into the transdermal drug delivery device, or a specific transdermal drug delivery device that is already configured or adapted to deliver the prescribed treatment regimen can be pulled by the pharmacist. In one embodiment, the user is then instructed to install a smartphone application and given a unique identification code by the prescribing healthcare provider. This unique identification code is entered into the smartphone application by the provider, thereby synchronizing any transdermal drug delivery devices provided with the unique user. The transdermal drug delivery device can then be provided to the user through the pharmacy. In one embodiment, the control unit can be provided by the provider or pharmacy and synchronized with the user's smartphone via a communication protocol (for example, wireless Bluetooth), and verified/authenticated by the unique identification code stored in an encrypted manner on the user's smartphone. Next, a limited quantity of drug cartridges can be provided to the user in a single package (for example, 7 drug cartridges for 7 days). The package may have an identification number or scannable barcode to be entered by the user into their smartphone, tablet computer, mobile device, or other hand held computing device, thereby linking the package to the user. Within the package of multiple cartridges, each individual cartridge may also be packaged individually, with a unique identification number or scannable barcode, again to be linked or scanned by the user prior to opening. After opening the package, the user attaches the device to their skin and completes any biometric confirmation or digital authentication via unique identification number or barcode on the drug delivery device itself, followed by initiation of the opioid drug delivery protocol. The transdermal drug delivery device then dispenses the opioid agonist to the patient in accordance with the prescribed regimen. Each step of this process is monitored by the smartphone application and recorded in a cloud-based database in an encrypted manner. This data can then be viewed on-demand by the prescribing physician, in order to document the time of use of each transdermal device and compliance with the prescribed dosing regimen. In one embodiment, the drug is only dispensed once the biometric confirmation or digital authentication is complete and verified via the smartphone application, thereby monitoring or preventing misuse or abuse. In another embodiment, the compliance or lack of compliance by the user is communicated to pre-selected individuals, via smartphone messaging or other means, in order to support adherence to the dosing regimen (for example, the user's healthcare provider, family members and friends, or to the user himself or herself). The absence of the system logging that the prescribed drug delivery cartridges were used within the dosing regimen time window may also indicate that the drug might have been diverted for abuse. The transdermal drug delivery device can be worn for the duration of the treatment, e.g., 7-30 days. At the end of the treatment the transdermal drug delivery device can be discarded or returned to the pharmacy for refurbishment. The tapered drug delivery treatment regimen described herein should minimize or eliminate withdrawal symptoms while providing effective acute or chronic pain management. The lack of the use of pills or single-use patches decreases the chance for abuse. The tapered delivery profile eliminates the harsh step downs associated with decreasing the dosage with pills and associated withdrawal symptoms often encountered when stepping down dosage amounts with pills.

US 2014/0100241 discloses providing different drug dosages to patients with lozenges, provided at a kiosk. The methods and devices disclosed herein can provide the entire opioid treatment protocol with a single drug delivery device or with multiple replaceable cartridges and do not require multiple trips to pick up medication. The methods and systems described herein also have the benefit of the patient not knowing the specific amount and time of the dosing of the opioid, which decreases the likelihood of withdrawal symptoms and decreases the probability of an addiction developing.

A variety of different drug delivery treatment regimen are provided herein that can be used with the transdermal drug delivery devices described herein. The transdermal drug delivery devices allow for a precise amount of drug to be delivered to the skin of the patient with a pre-programmed drug dosing regimen.

A tapered drug delivery regimen can include a high first opioid agonist dose followed by a series of decreasing opioid agonist doses. The drug delivery treatment regimen can have a smooth tapered profile, in contrast to the binary stepdown associated with pill based treatments that can enhance withdrawal symptoms and other physiological responses associated with opioid weaning. Weaning a patient off of an opioid can be difficult, especially a patient addicted to opioids. This is especially true for eliminating the last 20% or so of the opioid consumption. Typically the opioid consumption when provided with blind doses can be decreased from the initial amount down to about 20% of the initial amount gradually without the patient experiencing extreme or harsh withdrawal symptoms. The weaning of the last 20% of the opioid dosage can result in additional withdrawal symptoms occurring in the patient. The present disclosure proposes more slowly and smoothly tapering the opioid delivered to the patient to wean the patient off of the last 20% of the opioid. Extending the time to wean the patient off of the opioid can reduce the likelihood of withdrawal symptoms and in some cases the likelihood of a relapse.

In some embodiments the dose-tapering treatment regimen can include opioid doses decreasing by a consistent percentage over the previous dose. For example, the second dose and first dose can define a first percentage decrease based on a difference in the first dose and second dose divided by the first dose. The third dose and second dose can define a second percentage decrease based on a difference in the second dose and third dose divided by the second dose. In some embodiments, the first percentage decrease is equal to or greater than the second percentage decrease. In other embodiments, the first percentage decrease is equal to or less than the second percentage decrease.

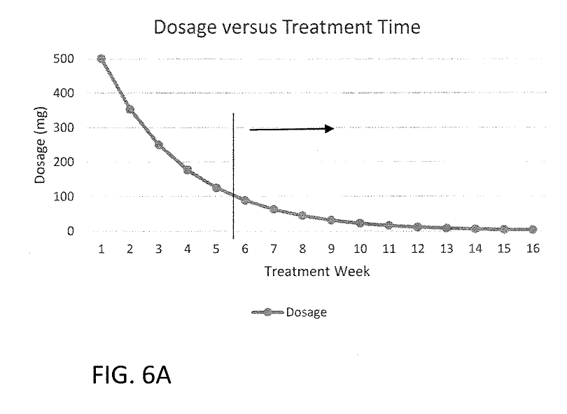

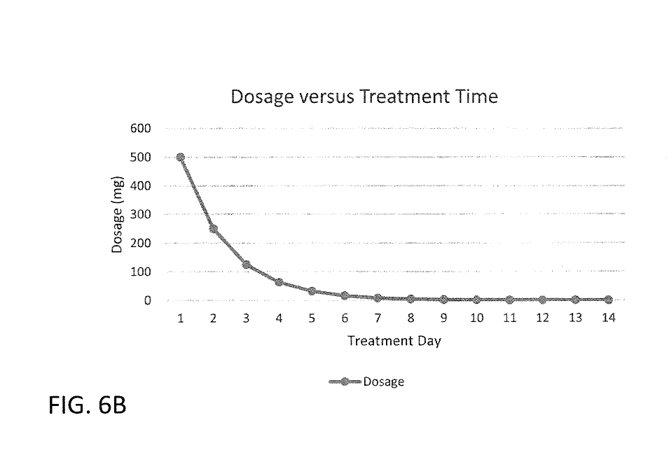

In some embodiments, the drug delivery treatment regimen can have an asymptotic shape or an asymptotic drug delivery profile. In one example, the dosing is proportional to 1/time. FIGS. 6A and 6B illustrate an example of an asymptotic dose-tapering profile that can be used with the methods and systems described herein. FIGS. 6A and 6B start with an arbitrary initial dosage of 500 and a treatment period of 16 weeks to taper the opioid delivery. The drug dosing scale and therapy week scales in FIGS. 6A and 6B are just one example. Many different opioid doses can be provided for a variety of different treatments and different therapy durations. The illustrated delivery profile has a longer tail to taper off of the last 20% or so of the initial opioid dosage. In some embodiments the asymptotic drug delivery treatment regimen includes each opioid dose decreasing over a previous opioid dose by an amount that is lower than a decrease in opioid dosage defined by the two preceding opioid doses.

In some embodiments the transdermal drug delivery can be in line with established guidelines or guidelines used by clinicians.

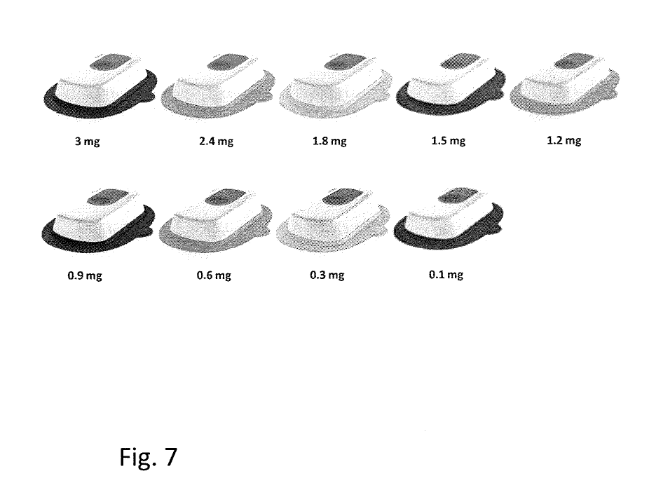

FIG. 7 illustrates transdermal drug delivery devices for delivering different doses of an opioid agonist, such as fentanyl, in accordance with some embodiments. FIG. 7 shows nine different dosing ranges for transdermal drug delivery devices, including dosing ranges of 3 mg, 2.4 mg, 1.8 mg, 1.5 mg, 1.2 mg, 0.9 mg, 0.6 mg, 0.3 mg, and 0.1 mg. The dosing can be stepped down using the different transdermal drug delivery devices as part of the tapering. Additional dosing ranges may include 25 mg, 20 mg, 15 mg, 10 mg, 5 mg, 4 mg, 3 mg, 2.4 mg, 1.8 mg, 1.5 mg, 1.2 mg, 0.9 mg, 0.6 mg, 0.3 mg, 0.2 mg, 0.1 mg and 0.05 mg, which allows for stepping down each dose by 20%-50%, with varying maintenance period spent on each dose.

In one example, fentanyl is provided by the transdermal drug delivery device. The transdermal drug delivery device provides an initial daily dosage level. The daily dosage is decreased until the dosage of fentanyl is reduced to about 3 mg/day. After decreasing the dosage to about 3 mg/day the dosage can be decrease by about 0.6 mg/day every ten to fifteen days to about 1.8 mg/day. After reaching a daily dosage level of about 1.8 mg/day the dosage can be decreased about 0.3 mg/day every ten to fifteen days.

In another example, the daily dose is decreased by 20%-50% per day until the dosage of fentanyl is reduced to about 0.5 mg/day. After reaching a daily dosage level of about 0.5 mg/day, the dosage can be decreased by about 0.15 mg/day every 2-5 days.

In one example, buprenorphine is provided by the transdermal drug delivery device. The transdermal drug delivery device provides an initial daily dosage level. The daily dosage is decreased by about 15% to about 20% per day until the dosage of buprenorphine is reduced to about 5 mg/day. After decreasing the dosage to about 5 mg/day the dosage can be decreased by about 0.5 mg/day every two to five days.

In another example, the daily dose is decreased by 20%-50% every 10 days until the dosage of buprenorphine is reduced to about 2 mg/day. After reaching a daily dosage level of about 2 mg/day, the dosage can be decreased by about 0.2 mg/day every 2-5 days.

In yet another example, the daily dose is decreased by 20%-50% per day until the dosage of buprenorphine is reduced to 4-8 mg/day. After reaching a dosage of 4-8 mg/day, it can further be reduced by 16% every 3-5 days until the dosage of buprenorphine is reduced to 1.3-1.6 mg/day. From there on, the dosage is reduced by 0.33-0.67 mg/day every 3-5 days.

The drug delivery treatment regimen is tapered to minimize withdrawal effects. The patient does not know the amount of the opioid agonist provided to the skin in any of the first, second, third doses, and subsequent doses. The lack of knowledge of the decrease of the opioid dose can prevent the patient from thinking that they are not getting a large enough opioid dose, and it may diminish the patient's perception of pain. Lack of knowledge of the decreased dosage can also be beneficial based on a perceived placebo effect provided by the patient knowing they are receiving the opioid but not realizing that the dosage level was decreased over the previous dose.

A variety of different drug formulations can be used herein. The drug formulation includes an opioid agonist or partial opioid agonist. Examples of opioid agonists include fentanyl, morphine, oxycodone, hydrocodone, hydromorphone, tramadol, oxymorphone, alfentanil, sufentanil, methadone, buprenorphine (partial agonist), etc. The opioid agonist in the transdermal drug delivery device can be provided in a solution with a biocompatible solvent.

In some embodiments an opioid antagonist or modified opioid antagonist can be optionally included with the transdermal drug delivery device. In one example, the modified opioid antagonist source can be provided in a solution with the opioid agonist. In another example, the opioid antagonist source or modified opioid antagonist can be provided in a container separate from the opioid with the container being adapted to break under a tampering force to mix the opioid agonist and the opioid antagonist source or modified antagonist. When the opioid antagonist is provided in a solution with the opioid agonist, the formulation and specific version of the opioid antagonist and opioid agonist can be selected such that the opioid agonist/agonist source is absorbed transdermally while the modified opioid antagonist is not absorbed through the skin. The opioid agonist source cannot be easily separated from the solution with the opioid antagonist or modified opioid antagonist. If the solution containing both the opioid agonist source and the opioid antagonist is injected or exposed to a mucous membrane, then the opioid antagonist will prevent the abuser from experiencing a high or euphoric effect of opioid agonist. The opioid antagonist and opioid agonist can be selected such that they are both stable in solution together. For example, the opioid agonist can be highly water soluble, in an ionic form, or have a high solubility in another solvent like alcohol-based solvents.

The devices and methods disclosed herein can reduce the likelihood of a person other than the prescription holder receiving the prescribed opioid. For example, the devices and methods disclosed herein can include a patient verification module that can be used to verify a patient biometric parameter to confirm that the patient's biometric parameter matches the patient to which the opioid treatment has been prescribed. Verifying the patients' biometric parameter can greatly reduce the likelihood of a non-authorized person using the transdermal drug delivery device to receive opioids.

In some embodiments of transdermal drug delivery devices, the biometric identification module can be configured to determine a patient's biometric parameter. Examples of sensors and systems that can be used in the biometric identification module include one or more of: a pulse oximeter, fingerprint scanner, heart rate sensor, ECG (Electrocardiogram) sensors, skin sensors, temperature sensor, blood flow sensor, impedance sensor, retina scanner, voice activation or recognition, and facial recognition system to verify the patients' biometric parameters. In some embodiments, the patients' biometric parameter can be verified using just one of the different biometric parameter modalities, such as in one example a facial recognition system. In other embodiments a combination of the different biometric parameter modalities can be used to confirm the patient identity and that the transdermal drug delivery system is in contact with the skin of the patient that has been prescribed the opioid. For example, a fingerprint scanner can be used to verify the patient identity and a combination of skin sensors can be also be used to confirm that the transdermal drug delivery device is in contact with the skin of the patient that has been prescribed the opioid. The patient identity is verified prior to initiating the drug delivery protocol or regimen of the opioid.

In some embodiments the transdermal drug delivery devices can include an identification module configured to receive an authentication parameter. The identification module is adapted to receive the authentication parameter. The process controller of the transdermal drug delivery device can be adapted to verify the authentication parameter prior to delivery of the opioid agonist or partial opioid agonist. The authentication parameter can be a code associated with a disposable part of the transdermal drug delivery device. The authentication parameter can be a user specific alphanumerical code, symbol, image, or barcode.

In some embodiments verifying the patient identity can include receiving an authentication parameter. The authentication parameter can be a patient-specific authentication parameter. The methods can also include generating the patient-specific authentication parameter. The methods can further include providing the patient-specific authentication parameter to the patient. The patient-specific authentication parameter can be a user specific alphanumerical code, symbol, image, or barcode. The authentication parameter can be a code associated with a disposable part of the transdermal drug delivery device. The authentication parameter can be an alphanumerical code, symbol, image, or barcode.

Methods are also described herein including receiving an opioid treatment regimen for a patient; generating a patient-specific code for the patient; receiving the patient-specific code; and initiating a transdermal opioid delivery protocol with a transdermal drug delivery device based on the opioid treatment regimen. The methods can further include receiving the patient-specific code from the transdermal drug delivery device. The methods can further include receiving the patient-specific code from a wireless data transfer from a remote computer network or a smartphone application. The methods can further include receiving a code associated with a disposable drug cartridge of the transdermal drug delivery device. The code associated with the disposable drug cartridge is a code, image, or barcode. The methods can further include receiving a code associated with a packing of a disposable drug cartridge of the transdermal drug delivery device. The code associated with the packing of the disposable drug cartridge is a code, image, or barcode. The methods can further include verifying the patient identity by observing a patient biometric parameter with a biometric identification module of the transdermal drug delivery device. The methods can further include one or more parameters associated with a patient compliance to the transdermal opioid delivery protocol and/or the opioid treatment regimen. The methods can further include generating a compliance data based on the one or more parameters associated with the patient compliance to the transdermal opioid delivery protocol and/or the opioid treatment regimen. The methods can further include providing the compliance data to the healthcare provider. Providing the compliance data can include sending an electronic message, electronic notification, or providing a graphical user interface containing the compliance data. Providing the compliance data can include incorporating the compliance data into an electronic medical record for the patient.

The sensors described herein can be used to measure patient health as well to look for opioid withdrawal symptoms, opioid toxicity/overdose symptoms (e.g. slowed respiratory rate), and to observe other patient characteristics that are relevant to the treatment received and prescribed by the healthcare provider. For example, if indications of an overdose are detected, like a reduced heart rate or breathing rate, then the treatment can be discontinued along with a notification being sent to the healthcare provider, and in serious situations a notification to alert an emergency medical service provider.

The devices can be used to analyze a patient's biological parameter to determine a symptom associated with opioid toxicity in the patient. The patient's biological parameters can be read from any of the sensors onboard the transdermal drug delivery device. For example, the patient's biological parameters can be obtained from one or more of a pulse oximeter, heart rate sensor, skin sensors, temperature sensor, blood flow sensor, impedance sensor, and retina scanner of the transdermal drug delivery device. The symptom associated with opioid toxicity in the patient can include a change in a breathing pattern, change in a heart rate, or combinations thereof. The process controller on the transdermal drug delivery device can analyze the patient's biological parameters to determine the signs or symptoms associated with opioid toxicity, such as symptoms associated with an opioid overdose. The transdermal drug delivery devices can be configured to send an alert or notification wirelessly upon detection of the symptom associated with opioid toxicity in the patient to a healthcare provider or emergency medical professional. In some cases, the transdermal drug delivery device can provide a stimulus to the patient prior to sending the notification to a healthcare provider. Examples of stimuli include tactile feedback, an electrical shock, or auditory feedback. The stimuli can prompt the patient for a response. A lack of response from the patient to the stimuli can further confirm the need for a notification to be sent to a healthcare provider.

The methods for using the devices described herein can further include providing an opioid antagonist to the patient after detection of the symptom associated with opioid toxicity. Examples of the opioid antagonists include Naloxone or Naltrexone.

The process controller on the transdermal drug delivery device can run an algorithm to analyze any of the patient's biological data and patient's biometric data collected by the sensors on the transdermal drug delivery device. The algorithm can analyze the breathing rate, heart rate, changes in heart rate, detected changes in circulation, and combinations thereof to look for symptoms associated with opioid toxicity or an opioid overdose. The algorithm can determine if and when it may be appropriate to provide a stimulus, such as an auditory, tactile, or shock, to the patient to test for response. The algorithm can determine when it is appropriate to send a notification or alter to a healthcare provider or emergency medical professional. The algorithm can also determine if and when it may be appropriate to provide an opioid antagonist to the patient. The algorithm can also adapt or modify the drug delivery treatment profile based on the specific patient receiving the opioid treatment and any patient-specific biological data and/or patient's biometric data collected or received by the transdermal drug delivery device.

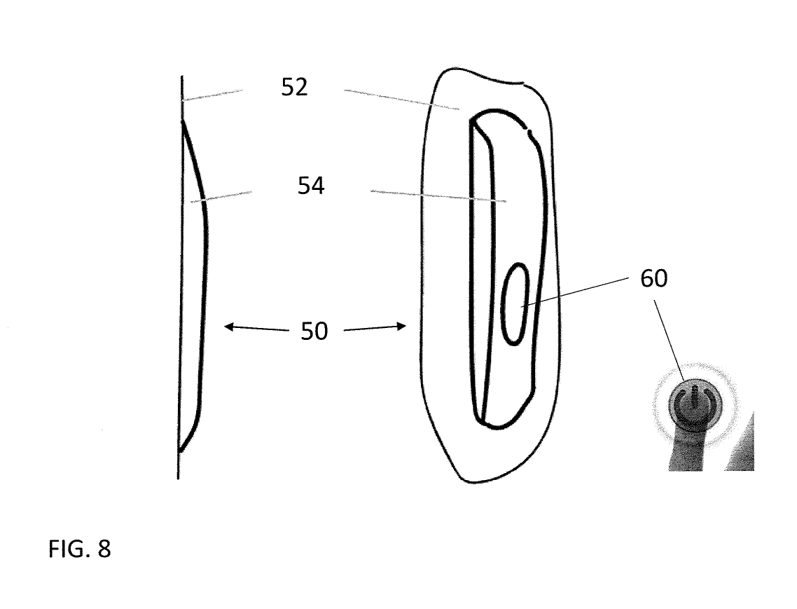

A fingerprint scanner can be included with the transdermal drug delivery devices described herein in some embodiments. The fingerprint scanner can be used to verify the patient identity and for biometric authorization of the patient. Fingerprints are unique to individuals and the fingerprint scanner can be used as a tool to authenticate the identity of individual prior to delivery of the drug formulation from the device. FIG. 8 illustrates an embodiment of a transdermal drug delivery device 50 for delivering an opioid with a fingerprint scanner 60 that can be used for patient identification. The device can initiate the drug delivery protocol after the patient scans their fingerprint with the scanner 60 and their identity is verified as the person with the prescribed opioid treatment.

A variety of sensors can be included in the transdermal drug delivery device. Sensors can observe physical or physiological parameters. Examples of physical sensors include mechanical sensors, thermal sensors, contract pressure sensors, electrical impedance sensors, and electrical capacitance sensors. Examples of physiological sensors include photoplethysmography sensors, ECG sensors, chemical sensors, and biological sensors.

Mechanical sensors can be incorporated into the transdermal drug delivery device. Mechanical sensors can be relatively simple to operate to receive a response when the desired mechanical condition is satisfied, such as pressure from skin contact.

Thermal sensors can be incorporated into the transdermal drug delivery device. For example a series of thermistors and a circuit can be used to measure the contact of the transdermal delivery device with the skin.

A contact pressure sensor can be incorporated into the transdermal drug delivery device. One or more contact pressure sensors can be used to determine the engagement of the transdermal drug delivery device with the skin of the patient.

An electrical impedance sensor can be incorporated into the transdermal drug delivery device. For example, the electrical impedance sensor can include a plurality of electrodes that send electrical current between the electrodes to provide an indication of the electrical properties between the patient engagement interface of the transdermal drug delivery device and the skin of the patient. The electrical impedance sensors could determine the contact between the device and the skin if the sensors generate a signal within a predetermined range of expected impedances.

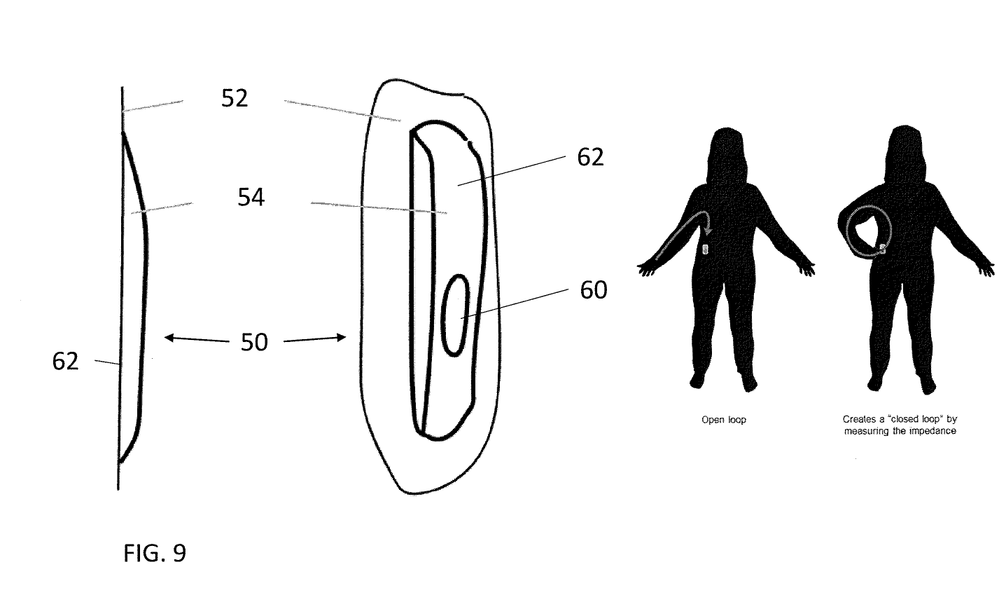

The transdermal drug delivery device can use an impedance sensor to measure impedance through skin to ensure that the patient is wearing the device at the time of delivery of the drug. Further, coupling impedance measurement with a fingerprint sensor, or any of the other sensors described herein, may be used to verify that the patient whose fingerprint is being used for authentication is actually wearing the device. FIG. 9 illustrates an embodiment of a transdermal drug delivery device 50 for delivering an opioid with a biometric sensor 60 along with a schematic example of biometric confirmation. The device illustrated in FIG. 9 includes impedance sensors 62 and a finger-print scanner 60. The device can confirm that the patient is wearing the device by reading the patient fingerprint and confirming that the scanned fingerprint is the same as the patient with the prescribed opioid treatment. The impedance sensors 62 can be used in combination with the fingerprint scanner 60 to confirm the patient identity while also measuring the impedance from the closed loop between the device on the patient's torso through the arm to the finger contacting the fingerprint scanner or other portion of the device with impedance sensor. For example, the outer part of the housing 54 could include an impedance sensor 62 or impedance could be measured through the finger print scanner 60.

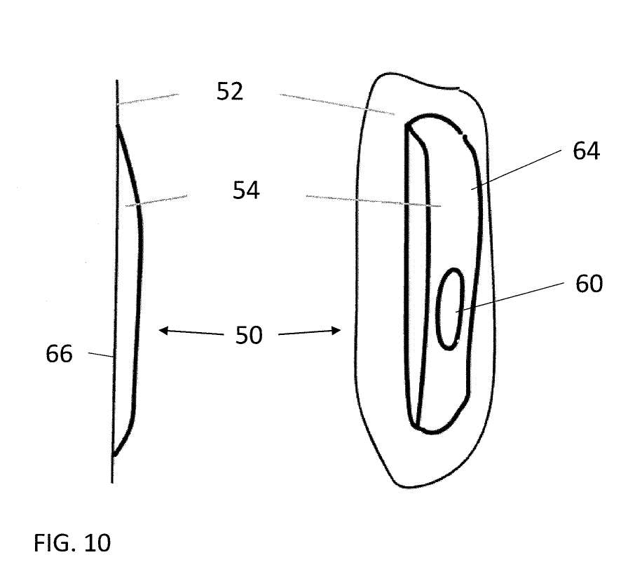

FIG. 10 illustrates an embodiment of a transdermal drug delivery device 50 for delivering an opioid that includes a fingerprint scanner 60, authentication button 64, and myographic sensor 66. The myographic sensor 66 can measure muscle movement and other properties of the wearer when the device is adhered to skin of the wearer. The authentication button 64 can be a pressure sensor or switch that can detect the pressing of the button 64.

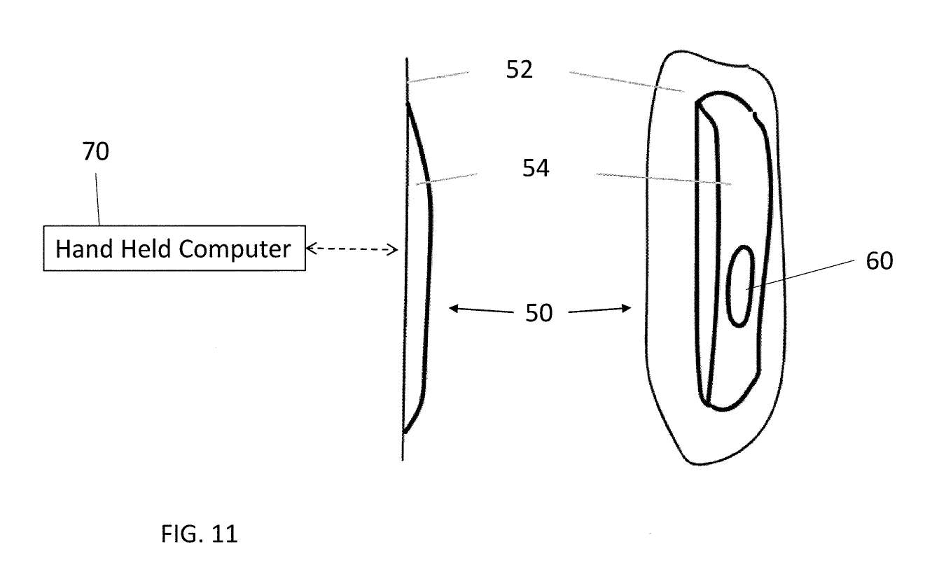

FIG. 11 illustrates an example of a transdermal drug delivery device 50 wirelessly sending data to a hand held computer device 70. Examples of hand held computer devices include smartphones, tablet computers, and the like. The wireless communication modules on the transdermal drug delivery device can wirelessly send and receive data. The data can be sent directly to the hand held computer device, over a remote network, or other wireless data transmission methods.

An electrical capacitance sensor can be incorporated into the transdermal drug delivery device. The electrical capacitance sensor can form one plate of a capacitor with the skin forming the other plate of the capacitor. The electrical capacitance sensors could determine the contact between the device and the skin if the measured capacitance produces a signal within a predetermined range of expected capacitance values.

Photoplethysmography sensors can be incorporated into the transdermal drug delivery device. Photoplethysmography sensors typically project light from a light source through the skin to a detector. The pulsatility and other properties of the blood can be observed with the detector. Multiple light waves can be provided to the skin to observe the blood oxygen saturation (e.g. 2 or more different wavelengths). Up to 8 discrete wavelengths of light can be used to interrogate carboxy hemoglobin (HbCO) in the blood. An example of a photoplethysmography sensor is a pulse oximeter.

A pulse oximeter and/or a heart rate sensor can be used in some embodiments of the transdermal drug delivery device. The pulse oximeter monitors the oxygen saturation of the patient's blood. Pulse oximeters are particularly helpful if the patient is undergoing respiratory depression because of overdose with opioids. Including a pulse oximeter in the device and a mechanism to inform the healthcare provider or care giver instantly is a useful tool to prevent deaths associated with abuse/overdose of opioids. The pulse oximeter can also measure the heart rate which further helps in elucidating if the patient is undergoing respiratory depression.

ECG sensors can be incorporated into the transdermal drug delivery device. The ECG sensors can be used to measure electrical activity associated with cardiac activity. The ECG sensors can provide information on the patient regarding the heart rate, heart rate variability, and other cardiac parameters. The ECG sensors can also be used to authenticate the patient or as part of the authentication process. The ECG sensors can be integral with the transdermal drug delivery device or in electrical or data communication with the transdermal drug delivery device.

Chemical/biological sensors can be incorporated into the transdermal drug delivery device. The chemical/biological sensors can analyze bio-chemical markers present in the body or skin. For example, perspiration can be analyzed to determine electrolyte content in a non-invasive manner. The presence of sodium (Na) or potassium (K) can indicate skin contact.

The sensors described herein can be used to sense contact with the skin. For example, skin sensors can be used to differentiate skin from other surfaces. The transdermal drug delivery device can initiate delivery of the active pharmaceutical ingredient after contact with the skin is determined with the sensor. The sensors can also monitor the temperature of skin as well as blood flow.

The transdermal drug delivery device can utilize a retina scanner to verify the patient identity. For example, a retina scanner can be used for biometric scanning to identify the patient prior to initiating the opioid drug delivery regimen. The retina scanner can be used prior to initiating the treatment and/or at regular pre-determined intervals. The retina scanner can be separate from the transdermal drug delivery device or included with the transdermal drug delivery device.

The transdermal drug delivery device can utilize facial recognition to verify the patient identity. For example a camera on the transdermal drug delivery device or a camera that is part of a mobile device (e.g. smartphone, tablet computer, etc.) can be used with advanced facial recognition software to verify the identity of the patient prior to initiating the drug delivery protocol. Facial recognition can be used prior to initiating the treatment and/or at regular pre-determined intervals.

The transdermal drug delivery device can include a sensor that recognizes vein patterns as the biometric indicator.

The transdermal drug delivery devices described herein can also include abuse-deterrence features. Examples of abuse-deterrence features include use of opioid antagonist, limiting direct access to the drug formulation, use of aversive agents, and rendering the drug formulation inactive in case of accidental or intentional breakage.

Examples of opioid antagonists include Naloxone, Naltrexone, Nalmefene, Samidorphan, and other opioid antagonists. The opioid antagonists can be modified as described herein to modify the properties of the opioid antagonists. The opioid antagonist can be incorporated with the transdermal drug delivery device in a variety of ways to deter abuse of the opioid agonist as described herein. Examples include the opioid antagonist mixed with the drug formulation containing opioid agonist, an opioid antagonist envelope adjacent to the drug formulation containing opioid agonist, an opioid antagonist mixed in with the construction material of the device, the device lined with opioid antagonist, etc.

In some embodiments of the transdermal drug delivery devices described herein, an opioid antagonist or a modified opioid antagonist is mixed in with the drug formulation containing opioid agonist. In this configuration, the opioid antagonist and drug formulation containing opioid agonist could be formulated such that the opioid antagonist does not penetrate through the skin and just the opioid agonist is delivered transdermally. The inclusion of the opioid antagonist with the drug formulation containing opioid agonist would deter an unauthorized user from ingesting the drug formulation containing the opioid antagonist because the antagonist would block the effects of the opioid agonist. The physicochemical properties of the opioid antagonist and opioid agonist can be matched, such as the hydrophobicity, log P value, etc. such that it is difficult to separate the opioid agonist from the opioid antagonist using common tampering methods.

The opioid antagonist or modified opioid antagonist can refer to an opioid antagonist that when taken by the patient is in an active state in vivo. The modified opioid antagonist can include a prodrug or a salt of the opioid antagonist. The modified opioid antagonist can be in a solution with the opioid agonist. The modified opioid antagonist can be in an inactive state in the transdermal drug delivery device in vitro but is configured to convert to an active state in vivo in systemic circulation in the patient. In some embodiments the modified opioid antagonist comprises a prodrug of Naltrexone or Naloxone. In some examples the prodrug of Naltrexone or Naloxone comprises a conjugation with poly ethylene glycol, an ester, or a carbonate.

In some embodiments the opioid antagonist is encapsulated in a polymer or inclusion complexes in the form of micro and/or nanoparticles. In some cases the opioid antagonist is in an isotropic mixture of oil, water, surfactant and/or co-surfactant. In some embodiments the opioid antagonist is dispersed in a lipophilic phase.

In some embodiments, micro-particles and/or nanoparticles can be used for the formulation including the opioid agonist and opioid antagonist. An alternate embodiment of the co-mixed formulation would have opioid agonist (e.g. fentanyl) in free-base form, and the free-base form or salt form of opioid antagonist encapsulated in inclusion complexes or micro-particles or nanoparticles formed with small molecules (e.g. cyclodextrins), and/or polymers (e.g. poly-ethylene glycol, poly lactic acid, poly glycolic acid, poly caprolactone, cellulose acetate phthalate, poly(lactic-co-glycolic acid), hydroxypropyl cellulose, hydroxypropyl methylcellulose, hydroxypropyl methyl cellulose phthalate, hydroxypropyl methyl cellulose acetate succinate, gelatin). These complexes/particles (micro/nano) typically have a hydrophobic center and hydrophilic surface, which may be used to maintain the opioid antagonist in a form that does not readily penetrate skin. The complexes/particles (micro/nano) may have opioid antagonist uniformly distributed in the polymer and is large enough to not permeate skin passively. Attempting to extract the formulation with common household solvents would alter the formulation solvent environment (pH, solubility of the polymer, etc.) resulting in opening up of these inclusion complexes/particles (micro/nano) and mixing of opioid antagonist with opioid agonist. Abusing the formulation by direct injection or other means would expose the abuser to both opioid agonist and antagonist, thus deterring abuse.

In some embodiments, the opioid formulation can include an inactive opioid antagonist (e.g. a modified opioid antagonist like a prodrug, drug-polymer conjugate etc.) that is adapted to be converted to an active form in-vivo within the patient. The opioid antagonist prodrug does not permeate the skin barrier, but can be converted to the active version in-vivo in case of formulation abuse (e.g. intravenous injection of formulation). Examples of such opioid antagonist prodrugs include an opioid antagonist (e.g. naloxone or naltrexone) conjugated with a short poly ethylene glycol chain via a breakable linkage such as an ester or carbonate linkage. An alternative approach to this embodiment includes antagonist drug conjugation with a biodegradable or biocompatible polymer. The conjugation may be via a breakable linkage such as an ester or carbonate linkage. Examples of biodegradable or biocompatible polymers include poly lactic acid, poly glycolic acid, poly caprolactone, cellulose acetate phthalate, poly(lactic-co-glycolic acid), hydroxypropyl cellulose, hydroxypropyl methylcellulose, hydroxypropyl methylcellulose phthalate, hydroxypropyl methylcellulose acetate succinate.

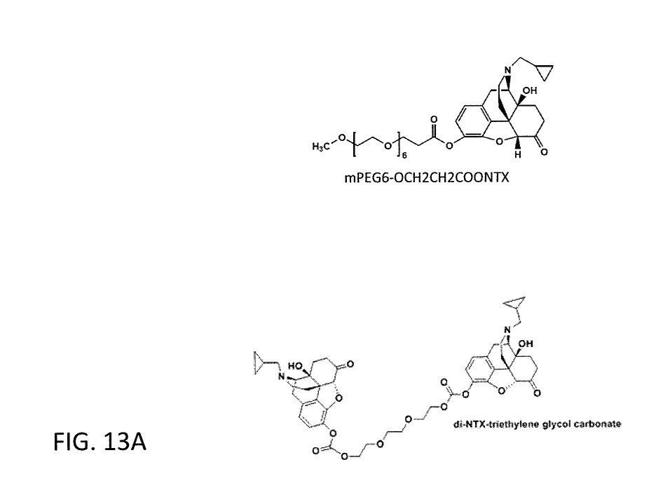

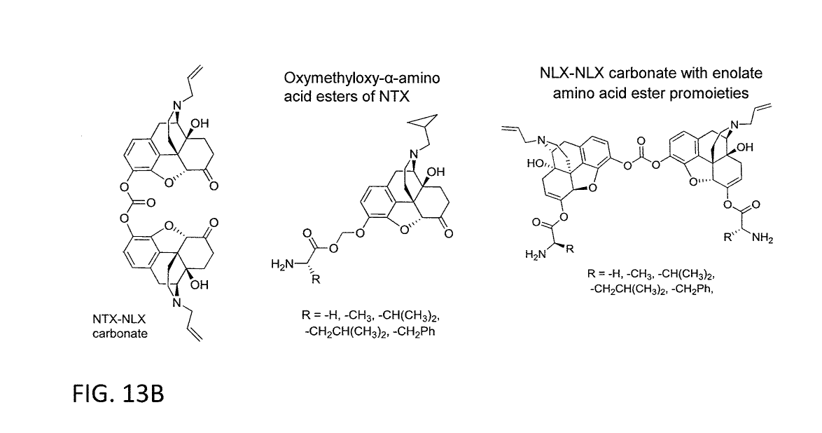

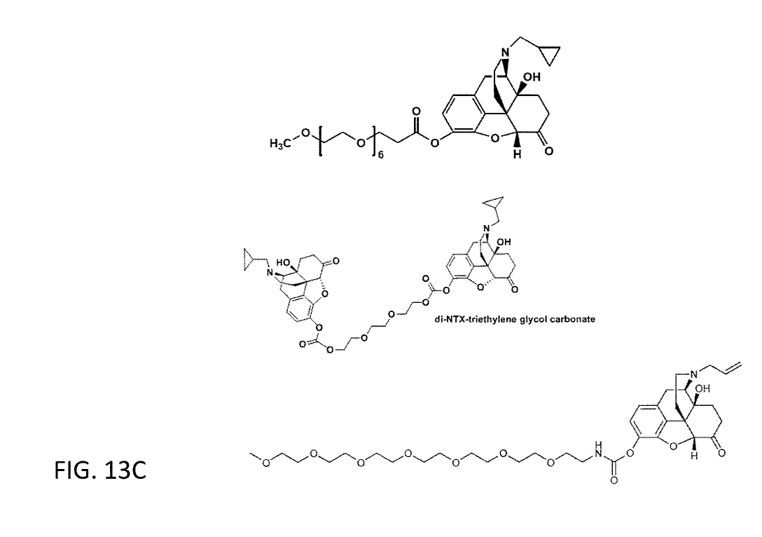

FIGS. 13A-13C illustrate chemical formulas of Naltrexone (NTX) and Naloxone (NLX) with various conjugations that can be used in the transdermal drug delivery systems described herein. Naltrexone and naloxone were modified to increase the molecular weight of the prodrug formulation to reduce or inhibit skin permeation. When the formulation is properly used the naltrexone or naloxone prodrug does not substantially permeate the skin of the patient while the opioid agonist permeates the skin. If the formulation containing the opioid agonist and the naltrexone/naloxone prodrug is injected then the naltrexone/naloxone prodrug will be rapidly released to block the effects of the opioid agonist. FIG. 13A illustrates examples of Naltrexone prodrugs with PEG conjugations. FIG. 13B illustrates a Naltrexone/Naloxone carbonate, a Naltrexone with an amino acid conjugation, and a Naltrexone/Naloxone with enolate amino acid ester promoieties. FIG. 13C illustrates examples of Naltrexone prodrugs with PEG conjugations.

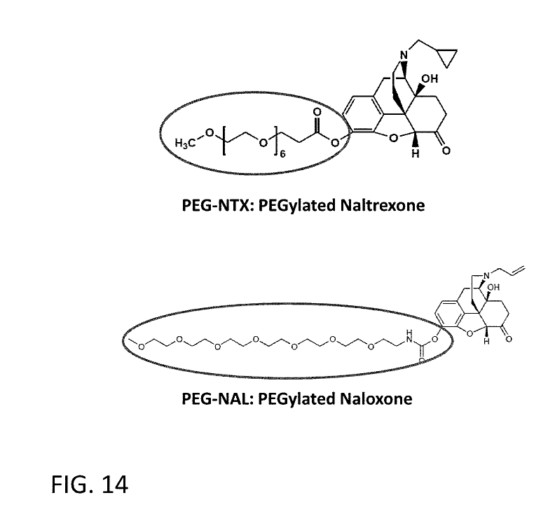

FIG. 14 illustrates chemical formulas of Naltrexone and Naloxone conjugated with poly ethylene glycol (PEG) that can be used in the transdermal drug delivery systems described herein. Tests showed that the PEG conjugated Naltrexone and Naloxone were converted to the free drugs in blood plasma. The PEG conjugated Naltrexone and Naloxone were incubated at 37° C. in pooled human plasma. Samples were withdrawn at 0 minutes, 30 minutes, 60 minutes, and 120 minutes. The samples were analyzed using HPLC-UV. The formation of the free drugs was determined at two hours to be 27% for PEG conjugated Naltrexone and 38% for PEG conjugated Naloxone.

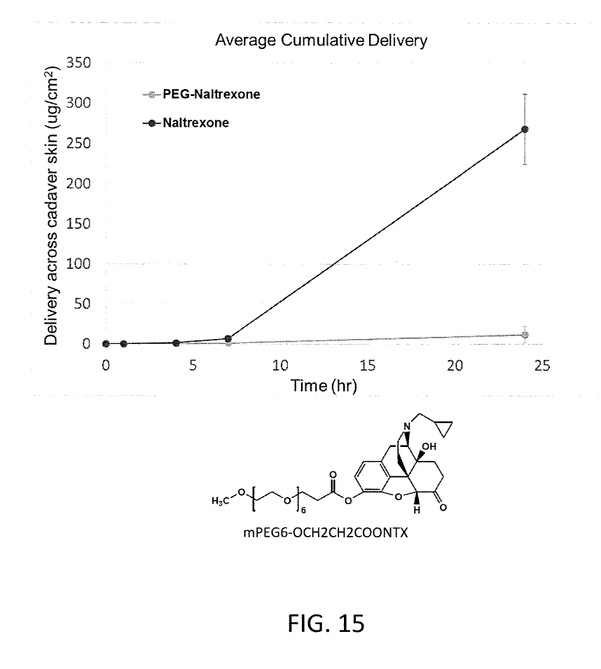

FIG. 15 is a graph illustrating the average cumulative delivery across a cadaver skin for Naltrexone and PEG conjugated Naltrexone. The prodrug of Naltrexone with the PEG conjugation increased the molecular weight of the compound to be about 690 g/mole. The graph shows that the flux across the cadaver skin was much lower for the PEG conjugated Naltrexone than the Naltrexone. The average flux for the Naltrexone was about 15.37 micrograms (μg) per hour per cm². The average flux for the PEG conjugated Naltrexone was about 0.62 micrograms (μg) per hour per cm².

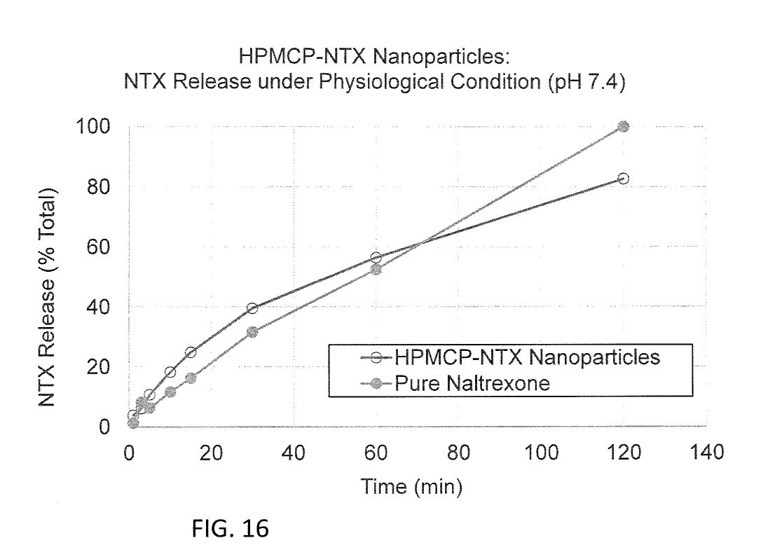

FIG. 16 is a graph comparing the release profile for pure Naltrexone versus Naltrexone released from nanoparticles in accordance with some embodiments. The nanoparticles prevent or reduce the Naltrexone from crossing the transdermal membrane. If the nanoparticle Naltrexone formulation is injected or exposed to physiological conditions then the Naltrexone is released from the nanoparticles to block the effects of the opioid agonist. FIG. 16 shows that the drug release profile is similar between pure Naltrexone and the Naltrexone released from HPMCP nanoparticles.

Methods of delivering an opioid to a patient are also provided. The methods can include moving an active pharmaceutical ingredient comprising an opioid agonist disposed in an opioid source from the opioid source to a transdermal membrane of a transdermal drug delivery device; moving a modified opioid antagonist disposed in the opioid source from the opioid source to the transdermal membrane; and passing a first portion of the active pharmaceutical ingredient comprising the opioid agonist across the transdermal membrane.

Passing the first portion of the active pharmaceutical ingredient comprising the opioid agonist across the transdermal membrane can have a first flux rate. The modified opioid antagonist can have a second flux rate across the transdermal membrane at a second flux rate. The second flux rate can be about zero in some embodiments. In some embodiments the ratio of the second flux rate to the first flux rate is less than about 1:10. In some embodiments the ratio of the second flux rate to the first flux rate is less than about 1:25. In some embodiments the ratio of the second flux rate to the first flux rate is less than about 1:50. In some embodiments the ratio of the second flux rate to the first flux rate is less than about 1:100.

The methods can also include passing a second portion of the opioid agonist across a skin of a patient wearing the transdermal drug delivery device at a first absorption rate. In some embodiments the methods can also include passing a portion of the modified opioid antagonist across the skin of the patient wearing the transdermal drug delivery device at a second absorption rate. In some cases the second absorption rate is about zero. The ratio of the second absorption rate to the first absorption rate can be less than about 1:10. The ratio of the second absorption rate to the first absorption rate can be less than about 1:25. The ratio of the second absorption rate to the first absorption rate can be less than about 1:50. The ratio of the second absorption rate to the first absorption rate can be less than about 1:100.

In some embodiments a micro-emulsion can be used for the formulation including the opioid agonist and opioid antagonist. For example, the co-mixed formulation would include opioid agonist (e.g. fentanyl) in a free-base form and opioid antagonist (e.g. Naloxone, Naltrexone), in an isotropic mixture of oil, water, surfactant and/or co-surfactant (e.g. a micro-emulsion). The isotropic mixture would consist of droplets of hydrophilic opioid antagonist dispersed in the lipophilic phase containing dissolved lipophilic opioid agonist. Examples of surfactant are Tween 80, polyoxyethylene, lecithin, etc. The oil phase may consist of fatty acids, terpenes, vegetable oils, medium chain mono-, di- and triglycerides etc. Micro-emulsions are stable and clear formulations. However, tampering with the formulation would disrupt the droplets/micelles thereby releasing the opioid antagonist from the hydrophilic core, especially with attempts to extract the opioid agonist using common household solvents such as vinegar, alcohol, etc. Disruption of micro-emulsion will mix the opioid antagonist with opioid agonist and prevent abuse of the opioid agonist by the user via alternate routes. Alternately, droplets of hydrophobic opioid agonist would be dispersed in the hydrophilic phase containing dissolved hydrophilic form of opioid antagonist. Tampering with the formulation would result in disruption of microemulsion and mixing of opioid agonist with antagonist, thereby preventing misuse/abuse.

In some embodiments an opioid antagonist envelope can be provided adjacent to the opioid agonist source. In this embodiment, the opioid antagonist can be provided in the form of a solid or liquid formulation that encapsulates the container holding the opioid agonist/drug formulation. The opioid antagonist formulation could be arranged adjacent to or in the direct path required to access the drug formulation containing opioid agonist from most points of entry. Consequently, tampering with the drug cartridge or any attempt to extract the drug formulation containing opioid agonist via a needle or syringe would likely lead to the antagonist formulation mixing with the opioid agonist formulation and thus deter abuse.

In some embodiments, the opioid antagonist can be mixed in with the construction material of the transdermal drug delivery device. In this embodiment, the opioid antagonist is embedded or included in the material used to manufacture the drug formulation container such that tampering with the drug cartridge in an attempt to extract drug formulation containing opioid agonist via a needle or syringe would break the drug formulation container and lead to the antagonist contacting or leaching into the opioid agonist formulation thus deterring abuse.

In some embodiments, the device can be lined with opioid antagonist. In this embodiment, the device could contain a coating of the opioid antagonist on one or more inner surfaces of the transdermal drug delivery device. Tampering with the device would lead to the drug cartridge breaking and exposing the opioid agonist drug formulation to the opioid antagonist.

In some embodiments, the drug formulation contains opioid agonist and opioid antagonist and the transdermal membrane in the device is impregnated with selective moieties (e.g., opioid antagonist antibodies, sulfonic acid based polymer) that preferentially bind with opioid antagonist. The selective moieties filter opioid antagonist from the drug formulation prior to the formulation coming in contact with skin for opioid agonist delivery.

The transdermal drug delivery devices can also be designed to limit access to the drug formulation. The materials of construction of the transdermal drug delivery devices can be selected to withstand common tools used to tamper with medications. For example, the material used for construction of the drug cartridge can be chosen such that it cannot be easily broken into by using the means commonly available in a household. Examples of commonly available means include kitchen utensils (e.g., spoon, forks, knives, etc.), electric tools such as household drill-sets, coffee grinders, etc., use of common solvents (e.g., ethanol, acetone, etc.), heat treatment (e.g., stovetop or hot water), cold treatment (e.g., storing in freezer), alternating treatments of heat and cold (e.g., heating on stovetop, followed by storing in freezer), etc.

The transdermal device and/or drug container can also include multiple layers. For example, multiple layers with different/complimentary characteristics can be used to deter abusers from tampering with the transdermal drug device. For example, a sticky material can be sandwiched between two hard layer(s) on the outside to limit access to the drug formulation.

The transdermal drug delivery device can utilize small volumes for the drug formulation. For example, the drug formulation may be stored in multiple cells of small volume, thus making extraction of the drug in usable quantities difficult.

In some embodiments, an aversive agent can be included in the transdermal drug delivery device to discourage abuse of the opioid. An aversive agent can be considered as a substance that result in an unpleasant or adverse reaction or strong dislike when encountered. An aversive agent can be employed to deter abuse of the drug formulations containing the opioid. A variety of different aversive agents can be included with the transdermal drug delivery devices and/or drug formulations described herein.

One example of an aversive agent is a bitterant or bittering agents. Examples of strong bitterants include, for example dentanonium, sucrose octaacetate, quercetin, brucine and quassin. The bitterant can be used to make the drug formulation smell or taste bitter to discourage ingestion or inhalation of the drug formulation.

Another example of an aversive agent is a pungent agent. Pungent agents can produce an unpleasant pungent flavor with a strong, sharp smell or taste. Examples of pungent agents include piperine, capsaicin, and allicin. Pungent agents causes a burning sensation and make good candidates for deterring abuse of the drug formulation by inhalation and ingestion.

Other aversive agents can be used that produce uncomfortable results in the patient. For example, chemicals that result in unpleasant adverse effect can also be used as aversive agents. In one example niacin can be used to prevent overdosing by producing significant adverse/unpleasant effects. Niacin causes unpleasant effects such as warmth or flushing, itching, sweating and/or chills when injected, inhaled or ingested in high doses. Certain pharmaceutical ingredients that can be irritating if ingested or inhaled can be incorporated in the drug formulation as well, such as colloidal silicon dioxide, crospovidone, magnesium stearate, microcrystalline cellulose, polyethylene oxide and sodium lauryl sulfate.

The transdermal drug delivery device and/or drug formulation can also optionally include a metabolizing or deactivating agent. Examples include enzymes that can metabolize the active drug in vitro or oxidizing agents such as hydrogen peroxide that can be incorporated in the device in such a way that manipulation of device releases the drug formulation and the enzyme/oxidizing agent deactivates/degrades the drug.

The transdermal drug delivery device and/or drug formulation can also optionally include a deactivating/denaturing drug formulation. For example, a non-specific adsorbent material like activated charcoal can be used. Activated charcoal could be incorporated in the device in such a way that if there is any attempt to tamper with the device, the activated charcoal will adsorb all the drug rendering the drug inactive or difficult to extract in a usable quantity.

Activated charcoal can be incorporated in the device in several different ways. In one example, a bed of activated charcoal or activated charcoal patches can be integrated at the base of the device such that in the case of tampering the drug formulation is released in liquid form on to the bed/patch and is neutralized. In another example, a coating of activated charcoal can be used to encapsulate the breakable cartridge (e.g. cartridge made of breakable material such as glass) containing drug formulation in liquid form. An attempt to manipulate the device will lead to breakage of the cartridge and introduction of the activated charcoal into contact with the drug resulting in neutralization of the drug. In yet another, example a coating of activated charcoal can be used on the inner surface of the device. Tampering with the device would lead to exposure of drug formulation to the activated charcoal resulting in inactivation of the drug formulation.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A transdermal drug delivery device comprising:
an active pharmaceutical ingredient comprising an opioid agonist or partial opioid agonist disposed in an opioid source;
an opioid antagonist disposed in the opioid source;
a transdermal drug delivery membrane configured to contact a skin of a patient and to provide the opioid agonist to the skin of the patient;
a fluid communication pathway between the opioid source and the transdermal drug delivery membrane;
a patient engagement surface adapted to secure the transdermal drug delivery device to the skin of the patient; and
a process controller configured to control a delivery of the opioid from the opioid source to the transdermal drug delivery membrane.

2. The device of claim 1, further comprising: a biometric identification module configured to determine a patient biometric parameter.

3. The device of claim 2, wherein the biometric identification module includes one or more of: pulse oximeter, fingerprint scanner, heart rate sensor, ECG sensor, skin sensors, temperature sensor, blood flow sensor, impedance sensor, retina scanner, voice activation or recognition, and facial recognition system.

4. The device of claim 2, wherein the process controller is adapted to verify the patient biometric parameter prior to delivery of the opioid agonist or partial opioid agonist.

5. The device of claim 2, wherein the process controller is adapted to analyze a patient biological parameter to determine a symptom associated with opioid toxicity in the patient.

6. The device of claim 5, the process controller further configured to send an alert or notification wirelessly upon detection of the symptom associated with opioid toxicity in the patient.

7. The device of claim 1, wherein the opioid antagonist includes Naloxone, Naltrexone, Nalmefene, or Samidorphan.

8. The device of claim 1, wherein the opioid antagonist is in a container separate from the opioid source, wherein the container is adapted to break under a tampering force to mix the opioid agonist and the opioid antagonist.

9. The device of claim 1, wherein the opioid antagonist comprises a modified opioid antagonist.

10-14. (canceled)

15. The device of claim 9, wherein the modified opioid antagonist is present in nano-particles or micro-particles of a polymer or co-polymer.

16. The device of claim 15, wherein the polymer or co-polymer includes one or more of: cyclodextrins, poly-ethylene glycol, poly lactic acid, poly glycolic acid, poly caprolactone, cellulose acetate phthalate, poly(lactic-co-glycolic acid), hydroxypropyl cellulose, hydroxypropyl methylcellulose, hydroxypropyl methyl cellulose phthalate, hydroxypropyl methyl cellulose acetate succinate, and gelatin.

17. The device of claim 1, wherein the opioid antagonist is encapsulated in a polymer or inclusion complexes in the form of micro and/or nanoparticles.

18-21. (canceled)

22. The device of claim 1, the processor further configured to provide opioid agonist or partial opioid agonist to the patient according to a drug delivery regimen.

23. The device of claim 22, wherein the drug delivery regimen is patient-specific and preprogrammed into the transdermal drug delivery device by a healthcare provider.

24-41. (canceled)

42. The device of claim 1, wherein the opioid source, transdermal drug delivery membrane, fluid communication pathway, and patient engagement surface are disposed in a disposable part, wherein the process controller is disposed in a reusable part, wherein the reusable part and the disposable part are adapted to be removably engaged.

43. A method of delivering an opioid agonist to a patient comprising:
initiating a transdermal opioid delivery protocol;
providing a first dose of the opioid agonist or partial agonist to the skin of the patient from a transdermal drug delivery device, wherein the transdermal drug delivery device includes an opioid antagonist;
providing a second dose of the opioid agonist or partial agonist to the skin of the patient from the transdermal drug delivery device based on the transdermal opioid delivery profile; and
providing a third dose of the opioid agonist or partial agonist to the skin of the patient from the transdermal drug delivery device based on the transdermal opioid delivery profile.

44. The method of claim 43, wherein the drug delivery protocol includes a plurality of opioid doses.

45. The method of claim 43, wherein the drug delivery protocol comprises a treatment regimen with an asymptotic dose-tapering profile.

46. The method of claim 45, wherein the asymptotic dose-tapering profile includes each opioid agonist dose decreasing over a previous opioid dose by an amount that is lower than a decrease in opioid dosage defined by the two preceding opioid agonist doses.

47. The method of claim 43, wherein the drug delivery protocol includes a high first opioid dose and a series of decreasing opioid doses.

49-140. (canceled)
